(19) United States
(12) Patent Application Publication
(10) Pub. No.: US 2025/0138006 A1
(43) Pub. Date: May 1, 2025

(54) METHOD AND SYSTEM FOR THE MONITORING OF AN ANALYTE OF INTEREST

(71) Applicant: TECHNISCHE UNIVERSITEIT EINDHOVEN, Eindhoven (NL)

(72) Inventors: Menno Willem José PRINS, Eindhoven (NL); Rafiq Milan LUBKEN, Eindhoven (NL)

(21) Appl. No.: 18/683,656

(22) PCT Filed: Aug. 9, 2022
(86) PCT No.: PCT/NL2022/050462

§ 371 Date: Feb. 14, 2024

(30) Foreign Application Priority Data
Aug. 17, 2021 (NL) 2028978

(51) Int. Cl. G01N 33/557; G01N 33/53; G01N 33/543

(52) U.S. Cl. CPC G01N 33/557; G01N 33/5308; G01N 33/54313; G01N 33/54386; G01N 33/54393

(57) ABSTRACT

A method and biosensing system for monitoring an analyte by measuring the concentration of the analyte in a measurement chamber including an effective number of binding sites having a binding affinity to the analyte, wherein the measurement chamber has an effective volume in which the analyte has a significant probability to encounter the binding sites, and method includes providing a time-dependent sampling of the analyte, by providing a time-dependent exchange of analyte between a system and the effective volume of the measurement chamber, by performing at least one exchange modulation cycle including the steps: a) facilitating a primary exchange phase having a characteristic time of primary exchange and a duration of primary exchange, b) facilitating a primary-to-secondary switching phase having a characteristic primary-to-secondary switching time and a primary-to-secondary switching duration, and c) facilitating a secondary exchange phase having a characteristic time of secondary exchange and a duration of secondary exchange.

### TECHNICAL FIELD

The present invention relates to a method and a biosensing system for the monitoring of an analyte of interest, such as a chemical, biochemical, or biological substance or structure, present in or at a system of interest, such as a container, a reservoir, a reactor, a tube, a line, a vessel, a lumen, a tissue, an organ, or an organism.

### BACKGROUND

Biological systems and biotechnological processes exhibit time-dependencies that are at the most basic level controlled by the dynamics of the constituting analytes, such as small molecules, hormones, proteins, and nucleic acids. This calls for measurement technologies that allow the monitoring of analyte concentrations, for instance in order to serve fundamental research on biological and biomedical dynamics, to enable the development of patient monitoring strategies based on real time biomolecular data, as well as to enable the development of closed loop control strategies in biotechnological applications. Desirable characteristics of a generic monitoring technology are (1) sensitive and specific measurements, (2) small time delays between sampling input and data output, (3) short time intervals between successive measurements, and (4) a long total time span over which time-dependent analyte concentration data can be recorded.

It is a fundamental challenge to develop a technology that can rapidly monitor low-concentration analytes over long time spans. Sensitive measurement technologies are available, such as ELISA and flow cytometry, but these methods consume reagents for every sample taken and every concentration determined, which complicates applications where analyte concentrations need to be monitored over long time spans. On the other hand, sensing technologies that can operate without consuming reagents, such as surface plasmon resonance, redox cycling and quartz crystal microbalance, have not been designed for monitoring analytes at low concentrations, such as in the picomolar and sub-picomolar range.

A generic bioanalytical principle used to quantify analyte concentrations with high specificity and sensitivity, is the biochemical affinity of specific binding sites. The binding sites can be effectuated by binding materials (such as molecularly imprinted polymers or other nanomaterials) or by binder molecules (such as antibodies, aptamers, proteins, nucleic acids, and the like). Typically, binding sites are effectuated by binder molecules, with at least one binding site per binder molecule for binding to the analyte, where the binder molecules are mobile or immobilized. The specificity originates from molecular interactions such as charge, hydrogen bonding, van der Waals forces, and hydrophobic and steric effects. To be able to measure analytes at low concentrations with high sensitivity, binder molecules are needed that have strong interactions with their target analytes, which corresponds to high binding energies, low equilibrium dissociation constants K_{d}, and low dissociation rate constants k_{off}. However, this conflicts with the desire to have small time delays, because low dissociation rate constants would imply a need for long incubation times to reach equilibrium. Furthermore, low dissociation rate constants result in a slow reversibility, which conflicts with the wish to enable short time intervals between successive measurements.

### DESCRIPTION OF THE INVENTION

The present invention relates to a method and a biosensing system that enables rapid monitoring of low concentrations of analytes. The method is based on the use of binder molecules with a high affinity in a limited-volume assay, with a reversible detection principle and time-controlled sampling of the analyte of interest. The system allows optimal tradeoffs between time characteristics and sensitivity. The present invention presents the measurement concept, time-dependencies of sensor signals, and a comprehensive analysis of the achievable time characteristics and sensitivity as a function of sensor design parameters. It was found that the sensing methodology enables precise and accurate quantification of low analyte concentrations, with time delays and interval times that are much shorter than the time dictated by the dissociation rate constant of the binder molecules. Furthermore, due to the reversible detection method, measurements can in principle be done over an endless time span.

In order to provide such a rapid monitoring of low concentrations of analytes, the present invention provides hereto a method for the monitoring of an analyte of interest, such as a chemical, biochemical, or biological substance or structure, present in or at a system of interest, such as a container, a reservoir, a reactor, a tube, a line, a vessel, a lumen, a tissue, an organ, or an organism, wherein a fluid or another viscoelastic medium or material comprises the analyte of interest, by measuring the concentration of the analyte of interest in a measurement chamber, wherein the measurement chamber comprises an effective number of binding sites (N_{b}), wherein the binding sites have a binding affinity to the analyte of interest, wherein the measurement chamber has an effective volume (V_{ch}) in which the analyte of interest has a significant probability to encounter the binding sites, and wherein the method comprises the step of providing a time-dependent sampling of the analyte of interest, by providing a time-dependent exchange of analyte between the system of interest and the effective volume (V_{ch}) of the measurement chamber, by performing at least one exchange modulation cycle comprising the following successive steps:
a) facilitating a primary exchange phase having a characteristic time of primary exchange (τ_{pr.exch.}) and a duration of primary exchange (t_{pr.exch.});
b) facilitating a primary-to-secondary switching phase having a characteristic primary-to-secondary switching time (τ_{pr.sec.switch}) and a primary-to-secondary switching duration (t_{pr.sec.switch}); and
c) facilitating a secondary exchange phase having a characteristic time of secondary exchange (τ_{sec.exch.}) and a duration of secondary exchange (t_{sec.exch.}),
wherein the exchange modulation cycle is repeated for any time-dependent sampling further provided, and wherein:
   the number of binding sites (N_{b}) and/or the effective volume (V_{ch}) of the measurement chamber is selected such that the effective volumetric binding site concentration (C_{b,ch}) in the measurement chamber is present in excess compared to the effective equilibrium dissociation constant (Kd) of the affinity binding between analyte of interest and binding sites, where C_{b,ch} is expressed as N_{b}/V_{ch};
   the concentration of the analyte of interest is determined by direct or indirect measuring the time-development of the amount of analyte of interest bound to at least one or more binding sites; and
   the direct or indirect measuring of the time-development of the amount of analyte of interest bound to at least one or more binding sites involves at least two measurements performed at different time-points in at least one exchange modulation cycle.

It is noted that the terms “direct measuring” or “indirect measuring” in relation to the amount of analyte of interest bound to at least one or more binding sites relate, respectively, to the direct measuring of the analyte of interest or the indirect measurement of the analyte of interest by measuring an analyte-analogue or analyte derivative or another molecule or another substance or another object or a chemical or physical property related to the analyte.

In the method of the present invention, the binding sites may be present on or in a supporting structure, such as a planar surface, a surface with concave or convex structure, a chemically and/or physically patterned surface, a particle, a polymer, or a porous matrix. Alternatively or in addition to the supporting structure the binding sites may be present in said fluid or another viscoelastic medium or material comprising the analyte of interest.

The method of the present invention may further comprise:
the sum of the duration of primary exchange (t_{pr.exch.}) and the primary-to-secondary switching duration (t_{pr.sec.switch}) and the duration of secondary exchange (t_{sec.exch.}) is larger than a characteristic time-to-equilibrium (T) in the measurement chamber.

Further to the method defined above, the exchange modulation cycle may be repeated by performing an additional step d) after step c) comprising:
d) facilitating a secondary-to-primary switching phase having a characteristic secondary-to-primary switching time (τ_{sec.pr.switch}) and a secondary-to-first switching duration (t_{sec.pr.switch}), and, optionally, wherein:
   the sum of the duration of primary exchange (t_{pr.exch.}) and the primary-to-secondary switching duration (t_{pr.sec.switch}) and the duration of secondary exchange (t_{sec.exch.}) and the secondary-to-primary switching duration (t_{sec.pr.switch}) is larger than a characteristic time-to-equilibrium (τ) in the measurement chamber.

As used herein, the terms “characteristic time of primary exchange” and “characteristic time of secondary exchange” refer to the time required to achieve 63% analyte exchange in the measurement chamber, i.e. the time required to evolve from a starting condition to a condition where a significant amount (63%) has been achieved of the change of concentration due to analyte exchange. Also, as used herein, the terms “characteristic primary-to-secondary switching time” and “characteristic secondary-to-primary switching time” refer to the time required to achieve 63% switching from primary to secondary phase and secondary to primary phase, respectively.

The term “binding sites” as used herein refers to “binders”, “binder molecules” or “binder materials”, which are able to bind and to form “analyte-binder complexes” with the analyte of interest.

The analyte of interest may be a chemical, a biochemical, or a biological substance or structure. The analyte of interest may be a supramolecular analyte, e.g. a virus particle, a supramolecular structure, a cell fragment, an intracellular body, an extracellular vesicle, a nanoparticle.

The time-dependent sampling of the analyte of interest according to the method of the present invention may be effectuated by time-dependent exchange of analyte by diffusion, advection, or by another active or passive physicochemical analyte transport method, or by a combination thereof.

In an embodiment of the method of the present invention, the duration of primary exchange (t_{pr.exch.}) may be smaller than the characteristic incubation time-to-equilibrium (τ). Alternatively or additionally, the duration of primary exchange (t_{pr.exch.}) may be larger than the characteristic time of primary exchange (τ_{pr.exch.}). The duration of primary exchange (t_{pr.exch.}) is preferably larger than one-hundredth of the characteristic time of primary exchange (τ_{pr.exch.}).

In a further embodiment of the method of the present invention, the primary-to-secondary switching duration (t_{pr.sec.switch}) may be larger than the characteristic primary-to-secondary switching time (τ_{pr.sec.switch}), and/or the characteristic primary-to-secondary switching time (τ_{pr.sec.switch}) may be smaller than the characteristic time-to-equilibrium (τ).

Also, in the case of step d), the secondary-to-primary switching duration (t_{sec.pr.switch}) may be larger than the characteristic secondary-to-primary switching time (τ_{sec.pr.switch}), and/or the characteristic secondary-to-primary switching time (τ_{sec.pr.switch}) may be smaller than the characteristic time-to-equilibrium (τ).

In an embodiment of the method of the present invention, the sum of the duration of primary exchange (t_{pr.exch.}) and the primary-to-secondary switching duration (t_{pr.sec.switch}) may be smaller than the characteristic time-to-equilibrium (τ).

In an embodiment of the method of the present invention, the duration of the secondary exchange (t_{sec.exch.}) may be smaller than the characteristic time of secondary exchange (τ_{sec.exch.}).

The at least one exchange modulation cycle may comprises two or more exchange modulation cycles, preferably at least three, four, five, six, seven, eight, nine, ten exchange modulation cycles.

In an embodiment, the at least one exchange modulation cycle comprises two or more exchange modulation cycles and wherein the measuring of the time-development of the amount of analyte of interest bound to at least one or more binding sites involves at least two measurements performed at different time-points in at least one exchange modulation cycle.

It is further noted that the facilitating of the phases during the at least one exchange modulation cycle may be performed by diffusion, advection, or by another active or passive physicochemical analyte transport method, or by a combination thereof. The phases of the at least one exchange modulation cycle may be effectuated by controlling the transport method in time.

It was further found that the increase or decrease of the time-development of the amount of analyte of interest bound to at least one or more binding sites during an exchange modulation cycle may depend on the amount of analyte of interest bound to at least one or more binding sites in said exchange modulation cycle and in a previous exchange modulation cycle.

In a further embodiment, the binding of the analyte of interest to a binding site is measured by:
a property of the analyte of interest, such as by charge, refractive index, fluorescence, luminescence, absorption, change of conformation, enzymatic activity, colour, or mass; or
a signal from another object, such as a molecule, substance, particle, label, surface, or a combination thereof, for example by energy transfer, resonance, scattering, absorption, motion, charge, refractive index, fluorescence, luminescence, change of conformation, enzymatic activity, colour, or mass,

wherein the measurement involves binding, conversion, competition, inhibition, displacement, amplification, molecular cascade, or sandwich formation, or a combination thereof.

The present invention further relates to a system for monitoring at least one analyte of interest, wherein the system comprises:
a measurement chamber comprising a number of binding sites (N_{b}), wherein the binding sites are able to bind the analyte of interest, and wherein the measurement chamber has an effective volume (V_{ch});
at least one exchange port, such as a tube, a channel, an opening, a connector, a valve, a permeable or semipermeable material, or a membrane, for time-dependent sampling of the analyte of interest involving transport into and/or out of the measurement chamber,

wherein the system is configured to perform the method according to the present invention and defined in the previous paragraphs.

The system of the present invention is preferably configured to monitor:
one analyte of interest; or
multiple analytes of interest, wherein the measurement chamber comprises multiple binding sites, and wherein each of the multiple binding sites is able to bind a specific analyte of interest selected from the group of multiple analytes of interest to be monitored.

In an embodiment of the present invention the system is configured to monitor multiple analytes of interest, and wherein the system is further configured to perform multiple methods according to the present invention in parallel, wherein each of the methods performed monitors one analyte of interest of the multiple of analytes of interest to be monitored.

In another aspect of the present invention, the invention relates to a biosensor device according to the present invention for use in in vivo biosensing, ex vivo biosensing, or in vitro biosensing, such as in, but not limited to, in vitro diagnostic testing, personal monitoring, animal testing, point-of-care testing, medical applications, life science applications, pharmaceutical applications, environmental testing, food testing, process monitoring, process control, water monitoring, environmental monitoring, air quality monitoring, vapor testing, breath fluid testing, chemical monitoring, forensics, biological, biomedical, or pharmaceutical research, agriculture, or to monitor assays with live cells, tissue, or an organ, organ-on-a-chip, or for measurement-and-control, closed loop control, real-time monitoring, and early warning applications.

The biosensor of the present invention may be used for continuous monitoring or for intermittent testing.

The analyte of interest may be measured continuously, i.e. by continuously taking samples for measuring, or non-continuously, i.e. by taking discrete samples for measuring.

The biosensor of the present invention may be prepared for immediate use, or rapid use, or plug-and-play use.

The biosensor may include transport methods such as diffusion, advection, acoustic excitation, magnetic actuation, thermal transport, convection, electrophoresis, optical excitation, syringe pumping, peristaltic pumping, membrane pumping, centrifugal excitation, actuation based on a fluid-fluid meniscus, actuation based on bubbles or droplets, ultrasonic excitation, actuation by electric fields, and the like.

The biosensor may be suited for multiplexing, i.e. measurement of several different analytes simultaneously or in parallel.

The biosensor may be used with a variety of binders, e.g. molecules, molecular constructs, and materials, such as, but not limited to, oligonucleotides, proteins, peptides, polymers, aptamers, small molecules, sugars, and molecularly imprinted polymers The analyte of interest may be a chemical, biochemical, or biological substance or structure. The analyte of interest may be measured directly in the system of interest, e.g. the measurement system may be provided in a flow path of the medium in the system of interest. Alternatively, a serial process may be applied, e.g. the medium may be sampled from the system of interest and may be transported to another system of interest, for example, the system of interest may be an organism, an organ, a tissue, a vessel, a cell system, a unit operation, a reactor, a lumen, a line, a tube, a bag, a receptacle, a chip, a well plate, an intermediate container, a reservoir, a chamber, a drip chamber.

The medium or sample may be pretreated in a sampling system, e.g. filtration, dilution, reagent addition, splitting, grinding, mixing, temperature treatment, heating, cooling, illumination, acoustic excitation, centrifugation, pressure application, under- or overpressure, cell lysis, enzymatic treatment, radiation treatment, amplification, separation, concentration, extraction, degassing, exposure to gas, removal of gas, and the like.

The biosensor may be provided with a functionality to provide wash steps, to add or release molecules or materials, to elute molecules or materials, or reset, regenerate or (re)activate the sensor or parts thereof.

### Further Embodiments of the Invention

In further embodiments of the device or method of the present invention, the biosensor may not be in direct contact with a system of interest, or may be in direct contact with a system of interest. Alternatively, the biosensor may be embedded or integrated or implanted in a system of interest. The biosensor can be placed at a distance from the system of interest. However, the biosensor may be located near the system of interest, on the system, wirelessly integrated, or the like. Samples can be put in a container and then transported to the biosensing system (sometimes called at-line or off-line operation), samples can be taken and automatically transported to the biosensing system (sometimes called on-line operation), or the biosensing system can be fully integrated with the system of interest (sometimes called in-line operation).

In further embodiments of the present invention, the device or method may be connected to or integrated in an industrial system or process, a fermenter, a bioreactor, an on-body device, a catheter, an in-body device, a wearable device, or an insertable device.

In a biosensing system with monitoring functionality, time-dependent samples can be taken, measurement data may be recorded, and a time profile may be established of analyte concentration. Also, a biosensor may be configured to receive a series of samples (from the same or from different sources) where the series of samples are serially or parallelly measured on the biosensor and result in time-dependent data that relate to different samples that have been supplied to the biosensor.

In further embodiments of the present invention, the device or method may be combined with a method or device module for sample pre-treatment or analyte pre-treatment, e.g. reagent addition, dilution, filtration, extraction, enrichment, purification, separation, amplification, change of buffer condition, stabilization, (dis)aggregation, or removal, modification, or addition of a chemical group or a biochemical domain or residue or moiety.

In further embodiments of the present invention, the device or method may be combined with a method or device module for optimization or control of operation, e.g. temperature, humidity, pressure, light conditions, vibration conditions, sound conditions, sterility, hygiene, ingress protection, cleaning, parts replacement, easy maintenance, calibration, and the like.

### DETAILED DESCRIPTION OF THE INVENTION

The basic concepts of the sensing methodology of the invention are sketched and exemplified in FIG. 1. The sensing system features time-dependent sampling of the analyte of interest, provided by a time-controlled analyte exchange between a biological or biotechnological system of interest and a measurement chamber (FIG. 1A). The measurement chamber contains specific binder molecules from which signals are recorded. Measurement time series are recorded and translated into concentration-time profiles, which should resemble as close as possible the true concentration-time profile of analytes in the system of interest. During the exchange of analytes, various processes occur, such as mass transport by advection and diffusion, and association and dissociation of analytes to binder molecules (FIG. 1B). A rectangular measurement chamber is assumed with height H, width W, and length L, resulting in an effective volume V_{ch} of the measurement chamber, where V_{ch}=HWL. The sensor surface is provided with binder molecules, where association and dissociation of analytes occurs. The binding process between analyte and binding sites can be described by an effective equilibrium dissociation constant (Kd), which represents a balance between on the one hand dissociation pathways that liberate binding sites or make them available, and on the other hand association pathways that occupy or block binding sites. To illustrate the concept of the invention, a simple bi-molecular system is assumed with reversible one-to-one binding between analyte (a) and binder (b): a+b⇄ab. The rates of association and dissociation depend on the association rate constant kₒₙ, the dissociation rate constant k_{off}, the density Γ_{b} of binder molecules, and the analyte concentration Cₐ at the sensor surface. These processes result in a time-dependent density γ_{ab} of analyte-binder complexes, also represented as a fractional occupancy f of binder molecules occupied by analytes: f=γ_{ab}/Γ_{b}. Variables γ_{ab} and f are changing as a function of analyte concentration and time. In an affinity-based sensor, the observed signal scales with f, therefore f is used in the present invention as the sensor read out parameter to determine the analyte concentration. Analyte exchange between the system of interest and the measurement chamber is facilitated by diffusion or a combination of diffusion and advection. A net molar flux Jₐ (orange gradient), caused by a concentration difference between the system of interest and the measurement chamber, facilitates diffusive mass transport of analytes between the measurement chamber and the system of interest. A developed laminar flow profile with flow rate Q and mean flow speed vₘ (black arrows) facilitates advective mass transport of analytes into the measurement chamber. Here it is assumed that diffusive transport occurs in both the longitudinal (x-direction) and the lateral direction (y-direction) and scales with the diffusion coefficient D, while advective transport occurs only in the longitudinal direction and scales with the mean flow speed vₘ.

FIG. 1C sketches two different sensor designs, namely an infinite-volume assay and a limited-volume assay. The graphs visualize the fractional occupancy f of binder molecules occupied by analytes as a function of time, with corresponding characteristic time-to-equilibrium τ, defined as the time needed to attain 63% of the difference between the starting level and the equilibrium level of f (see Supplementary Information 2). In an infinite-volume assay, continuous analyte exchange is enabled between the system of interest and the measurement chamber, where the system of interest is assumed to be much larger than the measurement chamber. The continuous analyte exchange could for example be facilitated by diffusive analyte transport across a contact area between the system of interest and the measurement chamber, while another configuration may involve a continuous flow of sample fluid, provided into the measurement chamber from the system of interest. When the analyte exchange is applied effectively and with negligible time delay, then the analyte concentration at the sensor surface (Cₐ) is equal to the input analyte concentration (C_{a,0}). In case of low analyte concentrations (C_{a,0}<K_{d}), the infinite-volume assay condition leads to a characteristic time-to-equilibrium τ≅1/k_{off} (see Supplementary Information 2). This implies that the time-to-equilibrium is determined by the dissociation rate constant k_{off}, so this time is long when the binder molecules strongly bind to the analytes.

The sensor design with a limited-volume assay has very different properties. Here, analyte exchange is limited, so that the binder molecules in the measurement chamber interact with only a limited sample volume and therefore with a limited amount of analytes. An effective volumetric concentration of binder molecules is defined C_{b,ch}=Γ_{b}/H=N_{b}/V_{ch}, where N_{b} is the number of binder molecules present in the measurement chamber. When C_{b,ch} is high, with C_{b,ch}>C_{a,0} and C_{b,ch}>K_{d}, then the time-to-equilibrium τ of the assay becomes dominated by the high concentration of binder molecules. When diffusional transport delays can be ignored, then the time-to-equilibrium of the assay equals τ≅1/(kₒₙC_{b,ch}) (see Table 1 and Supplementary Information 1 and 2). Thus, the time-to-equilibrium of the limited-volume assay is determined by the association rate constant and the effective volumetric concentration C_{b,ch} of binder molecules, which leads to equilibrium timescales that are much shorter than the time-to-equilibrium of the infinite-volume assay.

In monitoring applications, one wants to record measurements with one and the same sensor over long time spans. To realize the limited-volume assay principle in a monitoring application, the sensor needs to be switched between two different conditions: an open condition and a closed condition. In the open condition, analytes are exchanged effectively between the system of interest and the measurement chamber, as sketched in FIGS. 1A and 1B (see also Supplementary Information 5). In the closed condition, analytes are exchanged ineffectively between the system of interest and the measurement chamber, causing a limited-volume incubation in the measurement chamber, as sketched in the bottom graph of FIG. 1C. The switching concept is referred to between open and closed condition as “time-controlled analyte exchange” or as “providing a time-dependent exchange of analyte”. FIG. 1D illustrates the operating principle for a sensor where time-controlled exchange is realized by a modulated flow. Phase 1 is the primary exchange phase, where there is effective analyte exchange between the system of interest and the measurement chamber with duration t_{pr.exch}, due to a short characteristic time of primary exchange τ_{pr.exch}, so that the starting concentration in the chamber equals C_{a,0}. Phase 2 is the secondary exchange phase, where there is ineffective analyte exchange between the system of interest and the measurement chamber with duration t_{sec.exch}, due to a long a characteristic time of secondary exchange τ_{sec.exch}, so that the limited-volume assay condition is provided. During the secondary exchange phase, the analyte concentration Cₐ in the measurement chamber decreases over time (depletion) or increases over time (repletion), depending on the initial occupation of binder molecules fᵢₙᵢₜ by analytes, since analytes are exchanged ineffectively between the system of interest and the measurement chamber. When fᵢₙᵢₜ is low, the concentration of analytes in the measurement chamber decreases over time, corresponding to depletion of analyte. When fᵢₙᵢₜ is high, the concentration of analytes in the chamber increases over time, corresponding to repletion of analyte. It is assumed that there is no analyte exchange during the secondary exchange phase (i.e., τ_{sec.exch}→∞) and that the time-to-equilibrium is not influenced by the primary exchange phase (i.e., t_{pr.exch}<τ, see Supplementary Information 6). Therefore, for known fᵢₙᵢₜ, the supplied analyte concentration C_{a,0} can be derived from the measured time dependence of the fractional occupation f(t) during the secondary exchange phase. At least two measurements need to be done to determine the input analyte concentration C_{a,0}, for example a measurement at the initial value fᵢₙᵢₜ and a measurement at the final value f_{end}, as indicated in the graph. The time required to change from the primary to the secondary exchange phase, i.e., the primary-to-secondary switching phase, and the time required to change from the secondary back to the primary exchange phase, i.e., the secondary-to-primary switching phase, have a characteristic switching time τ_{switch} and a switching duration of t_{switch}. Here it is assumed that τ_{switch} and t_{switch} for both switching phases are negligibly small compared to the other time scales in the monitoring system.

By sequentially applying cycles with open condition and closed condition, discrete samples with a limited volume are serially measured and result in time-dependent data that relate to the different samples supplied to the sensor. The limited-volume condition ensures that C_{b,ch}>C_{a,0} and causes the binder molecules to influence the analyte concentration Cₐ in the measurement chamber. Each former measurement causes a varying nonzero initial fractional occupancy fᵢₙᵢₜ in the next measurement. The values of fᵢₙᵢₜ and C_{a,0} determine whether depletion or repletion occurs during the incubation phase. In case of depletion, a higher input analyte concentration C_{a,0} yields a larger, positive change of fractional occupancy Δf=f_{end}−fᵢₙₜ since more analytes are captured from solution, while for repletion a higher C_{a,0} yields a smaller, negative change of fractional occupancy Δf since less analytes are repleted from the sensor surface into solution. An important property of the sensor is that the interactions between binder and analytes are reversible. This gives the advantage that the limited-volume assay with time-controlled analyte exchange can be used over an endless time span.

It was further found that sensor design parameters influence the time characteristics and sensitivity of the sensing methodology. The time characteristics are quantified by finite-element simulations of mass transport in the sensor and reaction processes at the sensor surface, and the sensitivity is quantified by calculating the stochastic variabilities in the measurements. The simulations and calculations are verified by experiments using a sensing technique with single-molecule resolution, called Biosensing by Particle Mobility (BPM, see Supplementary Information 7).

FIG. 2 shows simulation results of the time-to-equilibrium of the limited-volume assay, for sensor designs with different chamber heights, different binder densities, and different flow rates, assuming standard parameter values as listed in Table 1. FIG. 2A shows how the time-to-equilibrium τ depends on the chamber height H, for a sensor with instantaneous analyte exchange (i.e., τ_{pr.exch}→0, see Supplementary Information 6 for the influence of analyte exchange on the sensor performance). The arrow on the x-axis indicates the height as listed in Table 1. The data show that the time-to-equilibrium increases with the chamber height. At small H, this increase in the time-to-equilibrium is caused by a decrease of the effective volumetric binder concentration C_{b,ch}=Γ_{b}/H, while at large H, this increase is caused by diffusive transport limitations. The inset shows the same data, plotted as a function of the Damköhler number (Da=τ_{D}/τ_{R,LV}=kₒₙΓ_{b}H/D); low Da means that the kinetics are limited by the reaction, high Da means that the kinetics are limited by diffusion. To achieve a fast time-to-equilibrium, the sensor should be designed with a large C_{b,ch}, so a small H.

FIG. 2B shows how the time-to-equilibrium depends on the binder density Γ_{b}, for a sensor with instantaneous analyte exchange. The arrow indicates the density as listed in Table 1. For small Γ_{b}, the time-to-equilibrium is long and determined by the dissociation rate constant (τ≅1/k_{off}). For Γ_{b}>HK_{d}≅20 μm⁻², the time-to-equilibrium decreases, until it stabilizes due to diffusive transport limitations (τ≅τ_{D}=H²/D). The inset shows the same data plotted as a function of Da. To achieve a fast time-to-equilibrium, the sensor should be designed with a large C_{b,ch}, so a large Γ_{b}.

FIG. 2C shows how analyte exchange by advection contributes to the time-to-equilibrium per measurement cycle. The primary exchange phase involves a temporary flow of fluid into the measurement chamber, with flow rate Q and duration t_{pr.exch} (see Supplementary Information 6 for the influence of analyte exchange on the sensor performance). In the simulations, t_{pr.exch} was chosen to be equal to the characteristic time of primary exchange τ_{pr.exch} which equals the characteristic advection time τ_{A}, so t_{pr.exch}=τ_{pr.exch}=τ_{A}=HLW/Q, which means that a total fluid volume is displaced equal to the volume V_{ch} of the measurement chamber. The time-to-equilibrium τ, which now includes a contribution t_{pr.exch} related to the exchange, is shown as a function of flow rate, for several values of the chamber aspect ratio λ=L/H. The arrow indicates the flow rate as listed in Table 1. For small Q the observed τ is limited by t_{pr.exch}, i.e., the advective transport of analytes from the inlet toward the point of sensing at a distance L/2 from the inlet, as sketched in FIG. 1D. For increasing λ, i.e., increasing L with a fixed H, the time-to-equilibrium increases since τ_{A} (and thus also t_{pr.exch.}) increases. For increasing Q, the time-to-equilibrium decreases, until it stabilizes at a level where the reaction and diffusion times determine the observed τ. The inset shows the same data (Da=2), supplemented with Da=0.2 (reaction-limited) and Da=20 (diffusion-limited), plotted as a function of the longitudinal Péclet number

$$( P {e}_{L} = {τ}_{D} / {τ}_{A} = \frac{Q}{λ D W} ) ;$$

low Pe_{L} means that the analyte exchange is limited by advection, high Pe_{L} means that the analyte exchange is limited by diffusion. A low Pe_{L} causes a long time-to-equilibrium due to slow mass transport by advection. Increasing Pe_{L} results in a decrease of the time-to-equilibrium due to rapid filling of the chamber, until it stabilizes at a τ value equal to the value indicated in FIG. 2A. FIG. 2C shows the flow rate required to minimize the influence of the primary exchange phase on the time-to-equilibrium. In the following sections, exchange with a high Pe_{L} is assumed, i.e., rapid filling of the measurement chamber, causing the time-to-equilibrium to be independent of the primary exchange phase.

FIG. 3 shows simulation results for a limited-volume assay with time-controlled analyte exchange. The analyte exchange is assumed to be instantaneous and the secondary exchange phase includes mass transport by diffusion and reaction kinetics within the measurement chamber itself, but no analyte exchange between the system of interest and the measurement chamber. FIG. 3A shows data for repeated incubations with C_{a,0}=0.1 pM. The analyte concentration Cₐ in the measurement chamber (brown line) and the fractional occupancy f of the binders by analytes (orange line) are plotted as a function of time, for conditions of analyte depletion (left) and analyte repletion (right). The time-to-equilibrium τ of each incubation equals approximately 340 s (see FIG. 2), having contributions from reaction (τ_{R}=200 s) and diffusion (τ_{D}=400 s). The contribution from the reaction to the time-to-equilibrium is much smaller than 1/k_{off}=10⁴ s, the value that would have been observed in case of an infinite-volume assay (cf. FIG. 1C). In absence of diffusion limitations, the acceleration that can be achieved with a limited-volume assay compared to an infinite-volume assay equals

$$α = \frac{1 / {k}_{off}}{{τ}_{R , LV}} = \frac{{k}_{o n} {Γ}_{b}}{H {k}_{off}} = {C}_{b , c h} / {K}_{d} ,$$

which clarifies how the speed of the assay is directly related to the ratio between effective volumetric binder concentration and the equilibrium dissociation constant.

FIG. 3B shows the response of a limited-volume assay with time-controlled analyte exchange where the sensor is incubated with an alternating analyte concentration (orange line): an analyte concentration below 0.1 pM (C_{a,0}=0.05 pM) and an analyte concentration above 0.1 pM (C_{a,0}=0.15 pM). The infinite-volume equilibrium fractional occupancy f_{eq,IV} (see Table 2) is given for C_{a,0}=0.15 pM and C_{a,0}=0.05 pM by the dashed black lines (see also Table 2). The panels on the right show zoom-ins of the sensor response at three different time periods (starting at t=0 h, 12 h, and 42 h). In all cases the time-to-equilibrium is τ=340 s=5.7 minutes. Incubation with C_{a,0}=0.15 pM gives depletion behavior at all times (since fᵢₙᵢₜ<f_{eq,IV}, top black line); for C_{a,0}=0.05 pM, depletion behavior is seen at t<10 h and repletion at t>10 h (when fᵢₙᵢₜ>f_{eq,IV}, bottom black line).

TABLE 1
Standard parameter values used in the finite-element
simulations. Details about the simulations are described in
Supplementary Information 4. Additional standard parameter
values are given in Table 2 (see Supplementary Information 1).

| | Parameter | Value | | Description |
|---|---|---|---|---|
| Input | H | 200 | μm | Measurement |
| | | | | chamber height |
| | D | 10⁻¹⁰ | m² s⁻¹ | Diffusion |
| | | | | coefficient |
| | | | | of the analyte |
| | Γ_{b} | 10⁻⁹ mol m⁻² | | Binder density |
| | | (600 μm⁻²) | | |
| | k_{off} | 10⁻⁴ | s⁻¹ | Dissociation rate |
| | | | | constant |
| | kₒₙ | 10⁶ | M⁻¹ s⁻¹ | Association rate |
| | | | | constant |
| | C_{a,0} | 0.1 | pM | Analyte |
| | | | | concentration |
| Derived | τ_{D} = H²/D | 400 | s | Characteristic |
| | | | | diffusion time |
| | $\text{} \begin{matrix}\begin{matrix}{τ}_{R , LV} = \\ \frac{1}{{k}_{on} {C}_{b , ch}} =\end{matrix} \\ \frac{H}{{k}_{on} {Γ}_{b}}\end{matrix}$ | 200 | s | Characteristic reaction time for limited-volume assay with C_{b,ch} >> C_{a,0} and C_{b,ch} >> K_{d} |
| | C_{b,ch} = Γ_{b}/H | 5 | nM | Effective |
| | | | | volumetric binder |
| | | | | concentration |
| | K_{d} = k_{off}/kₒₙ | 100 | pM | Equilibrium |
| | | | | dissociation |
| | | | | constant |

| | | |
|---|---|---|
| $\text{} \begin{matrix}α = \frac{{Γ}_{b}}{{HK}_{d}} = \\ {C}_{b , ch} / {K}_{d}\end{matrix}$ | 50 | Acceleration factor: reduction factor of the time-to- equilibrium of a limited-volume assay with τ(H, Γ_{b}), compared to an infinite-volume assay with τ(k_{off}). |
| Da = τ_{D}/τ_{R,LV} = | 2 | Damköhler number |
| kₒₙΓ_{b}H/D | | |

Note 1. How the size of analytes influences
the time-to-equilibrium of a limited-volume assay
Assume a sensor with standard parameter values given in Table 1 and
$Table 2 , then \frac{{C}_{b , ch}}{{C}_{a , 0}} = \frac{{Γ}_{b}}{{HC}_{a , 0}} = 5 \cdot {10}^{4} , indicating a condition of$
binder-excess. Here, three analyte exchange methods are considered:
instantaneous analyte exchange, analyte exchange by longitudinal
advection, analyte exchange by transverse diffusion. Two analyte sizes
are compared: small analytes, such as ions and small molecules (~0.1-1
nm, MW up to ~1 kDa) and large analytes, such as antibodies and
virions (~10-100 nm, MW between 100 kDa and 100 MDa).
Instantaneous analyte exchange (as in FIG. 2A): For small analytes with
D = 10⁻⁹ m² s⁻¹, Da = 0.2, the kinetics are reaction-limited (τ/τ_{R}~1) and
the time-to-equilibrium equals τ = 200 s (3 min). For large analytes with
D = 10⁻¹¹ m² s⁻¹, Da = 20, the kinetics are diffusion-limited (τ/τ_{R}~8) and
the time-to-equilibrium equals τ = 1,600 s (30 min).
Analyte exchange by longitudinal advection (as in FIG. 2C): Preferably
the sensor is designed with an analyte exchange process that hardly
contributes to the time-to-equilibrium. For small analytes with Da = 0.2,
no influence is observed at Pe_{L} > 10⁰ → Q > 10 μL min⁻¹. For large
analytes with Da = 20, no influence is observed at Pe_{L} > 10¹ → Q > 1 μL
min⁻¹.
Analyte exchange by transverse diffusion (as in Supplementary
Information 6, FIG. 8): According to FIG. 8, for small analytes the
analyte exchange process does not influence the time-to-equilibrium for
τ/τ_{R}~1 → τ = 200 s (3 min) while for large molecular complexes
τ/τ_{R}~20 → τ = 4,000 s (60 min). Large analytes cause a longer time-to-
equilibrium due to diffusion limitations in the reaction (see FIG. 2 and
FIG. 8). The kinetics can be improved by decreasing the chamber height
(see FIG. 2A). With H = 20 μm, large analytes give τ_{D} = 40 s, achieving
a 100× improvement in kinetics (see also Note 2).

FIG. 4 shows an experimental study on how the time-to-equilibrium in a limited-volume assay depends on the total binder concentration in the measurement chamber. Here, total binder concentration has two contributions, namely a contribution from surface-bound binders and a contribution from binders supplemented in solution. For detection use was made of Biosensing by Particle Mobility (BPM), which is an analyte monitoring principle with single-molecule resolution where the binding of analytes to specific binder molecules modulates the mobility of particles (see Supplementary Information 7). FIG. 4A shows a schematic representation of a measurement chamber with binder molecules present in the two forms: immobilized and non-immobilized. Immobilized binder molecules are present with an effective volumetric concentration C_{b,ch}. Binder molecules supplemented free in solution have concentration C_{b,suppl}. In absence of supplemented binder molecules (top), the total binder concentration in the measurement chamber equals C_{b,tot}=C_{b,ch}=Γ_{b}/H. In presence of supplemented binder molecules (bottom), the total binder concentration equals C_{b,tot}=Γ_{b}/H+C_{b,suppl}. Since the time-to-equilibrium of the reaction scales according to τ_{R,LV}∝1/C_{b,tot} (see Table 1), an increasing supplemented binder concentration C_{b,suppl} results in a smaller T. FIG. 4B shows the measured time-to-equilibrium τ (left) and the signal change ΔS (right) as a function of C_{b,suppl}, for an analyte concentration of 200 pM. The data show that the time-to-equilibrium decreases for increasing C_{b,suppl}. The measured signal change decreases with increasing supplemented binder concentration because only surface-captured analytes generate a measurable signal. The dashed lines in FIG. 4B represent model fits (see the caption), demonstrating a good correspondence between model and experimental results. It was found that the measurements of FIG. 4 prove the basic concept of the monitoring biosensor of the present invention, namely that a limited-volume design with time-controlled analyte exchange allows one to control the response time by tuning the concentration of binder molecules in the measurement chamber.

FIG. 5 shows how the analytical performance of the limited-volume assay depends on the sensor design. The results are based on numerical simulations with parameters as listed in Table 1. The analyte exchange is assumed to be instantaneous and the secondary exchange phase includes mass transport by diffusion and reaction kinetics within the measurement chamber only. All panels show curves for different values of the initial fractional occupancy fᵢₙᵢₜ of the binder molecules.

FIG. 5A shows the fractional occupancy of binders by analytes at the end of the incubation (fend) as a function of the input analyte concentration C_{a,0}. For fᵢₙᵢₜ=0 (dashed black line), fend scales linearly with the analyte concentration, which makes the sensor suitable for analyte quantification. For larger values of fᵢₙᵢₜ the curves start with a rather flat segment, from which one might erroneously conclude that under those conditions low analyte concentrations cannot be determined. Interestingly, the limited-volume assay has a linear dependence on concentration by focusing not on the absolute value of fend but rather on the change of fractional occupancy Δf (see Supplementary Information 2):

$$Δ f = {f}_{e n d} - {f}_{i n i t} = \frac{1}{α + 1} ( \frac{{c}_{a , o}}{{K}_{d}} - {f}_{i n i t} ) ≅ \frac{H}{{Γ}_{b}} ( {C}_{a , 0} - {K}_{d} {f}_{i n i t} ) \qquad ( Equation 1 )$$

This equation shows that Δf depends linearly on C_{a,0}, independent of the value of fᵢₙᵢₜ. This fact is also illustrated by the simulation results in FIG. 5B. The response scales linearly with concentration C_{a,0} and are down-shifted for increasing values of fᵢₙᵢₜ, in agreement with Equation 1 (note that the steep increase of the curves relates to the logarithmic x-axis). Positive values of Δf relate to depletion behavior and negative values to repletion. The curves cross the x-axis (Δf=0) when fᵢₙᵢₜ corresponds to the equilibrium condition, i.e., when there is no net association or dissociation during incubation because fᵢₙᵢₜ is equal to the equilibrium fractional occupancy of the infinite-volume case:

$${f}_{init} = {f}_{eq , IV} = \frac{{C}_{a , 0}}{{C}_{a , 0} + {K}_{d}} ≅ \frac{{C}_{a , o}}{{K}_{d}} .$$

For example, the curve for fᵢₙᵢₜ=10⁻³ crosses Δf=0 at C_{a,0}=fᵢₙᵢₜK_{d}=0.1 pM, as is highlighted in the inset of FIG. 5B.

FIG. 5C shows the precision of the concentration output of the sensor, i.e., the precision with which the analyte concentration in an unknown sample can be determined for a signal collection area of 1 mm². The precision is calculated based on Poisson noise, which gives the fundamental limit of the precision that is achievable with a molecular biosensor due to stochastic fluctuations in the number of analytes (see Supplementary Information 3 and 8). To calculate the precision, a sensor with initial fractional occupancy fᵢₙᵢₜ is provided with a sample with analyte concentration C_{a,0}, resulting in a Δf with variability σ_{Δf}, which via the slope of the calibration curve, given in FIG. 5B, leads to a variability σ_{C} in the concentration output of the sensor (see Supplementary Information 3). The precision is indicated as the concentration-based coefficient of variation CV_{C}=σ_{C}/μ_{C}, with σ_{c} the variability and μ_{c} the mean of the concentration output. FIG. 5C shows how the concentration precision depends on the analyte concentration and the initial fractional occupancy fᵢₙᵢₜ. For fᵢₙᵢₜ=0 (dashed line), the CV_{C} scales as 1/\sqrt{C_{a,0}}, in agreement with number fluctuations in a Poisson process (see Supplementary Information 3). For higher fᵢₙᵢₜ, a stronger dependency is observed (CV_{C}∝1/C_{a,0}) caused by the smaller relative change of the fractional occupancy (see Supplementary Information 3). The graph indicates the 10% precision level that is used to define the limit of quantification (LoQ) of the sensor. The results show that analyte concentrations in the sub-picomolar range can be measured with a precision better than 10%, even for high initial fractional occupancies.

FIG. 5D shows the precision of the concentration output of the sensor as a function of two design parameters, namely the measurement chamber height H (top panel) and the binder density Γ_{b} (bottom panel), at an analyte concentration C_{a,0}=0.1 pM, for an initial fractional occupancy fᵢₙᵢₜ between 0 and 0.01. The arrows indicate the height and density as listed in Table 1. For an increasing H, a decrease of CV_{C} is observed, caused by an increase in the number of analytes present in the measurement chamber. The CV_{C} is smallest for fᵢₙᵢₜ=0 and increases for increasing fᵢₙᵢₜ since the absolute change of fractional occupancy decreases. The CV_{C} decreases for increasing Γ_{b} caused by an increase in the number of analytes captured from solution. The CV_{C} reaches a plateau for fᵢₙᵢₜ=0 due to a limited number of analytes in the measurement chamber. For larger fᵢₙᵢₜ, the absolute change of fractional occupancy decreases and causes a less precise concentration determination; this effect is in particular visible at high Γ_{b} where the absolute number of analyte-binder complexes increases due to fᵢₙᵢₜ.

The tradeoff between precision and time-to-equilibrium is illustrated in FIG. 5E, for sensors with different heights of the measurement chamber (left) and different binder densities (right). The arrows indicate the time-to-equilibrium that results from the height and density as listed in Table 1. The left panel shows that an increase of H gives on the one hand a slower sensor response (due to a larger diffusion distance) but on the other hand a lower CV_{C} due to a larger number of analytes being exchanged by association and dissociation between the sensor surface and the measurement chamber. At low H, the CV_{c} strongly depends on fᵢₙᵢₜ due to the low number of analytes in the solution. The right panel shows again that the CV_{c} decreases for a slower sensor response, now controlled by decreasing the binder density Γ_{b}. At high Γ_{b} the time-to-equilibrium is diffusion-limited (resulting in τ=130 s). At low Γ_{b} the time-to-equilibrium is reaction-limited with τ=1/k_{off}=10⁴ (see FIG. 2B). At high Γ_{b}, the CV_{C} increases for increasing fᵢₙᵢₜ due to the larger amount of analytes on the sensor surface. At low Γ_{b}, the CV_{C} strongly increases due to the low number of captured molecules caused by analyte dissociation over the long incubation timescale.

Note 2. How the analyte size influences the sensitivity of the sensor.

Assume a sensor as given in Note 1. As suggested in Note 1, with H = 20
μm, large analytes give τ_{D} = 40 s, achieving a 100x improvement of
kinetics, but with a decrease in precision (see FIG. 5D). Using H = 20
μm, for small analytes this would result in CV_{c} = 1% and for large
analytes CV_{c} = 3%, but the precision decreases rapidly for increasing fᵢₙᵢₜ
(for example: fᵢₙᵢₜ = 10⁻³ → CV_{c} = 10% and CV_{c} = 100% for small and
large analytes respectively). The solution for this sharp decrease in
precision is to decrease the binder density Γ_{b} (see FIG. 5E, right) causing
an increased precision with an increase in the time-to-equilibrium as a
cost.

Given the above, the present invention provides a sensing methodology suitable for monitoring low-concentration analytes with high sensitivity, with small time delays and short time intervals, over an endless time span. The sensing methodology is based on a limited-volume assay, using high-affinity binders, a reversible detection principle, and time-controlled analyte exchange. Based on simulations it was studied how the kinetics of the sensor depend on mass transport and on the surface reaction in the measurement chamber, and how time-controlled analyte exchange determines the system response. Experimental results show the ability to control the sensor response time by tuning the total binder concentration in the measurement chamber. Finally, simulations show that the sensing principle allows picomolar and sub-picomolar concentrations to be monitored with a high sensitivity over long time spans.

Approaches described in literature for measuring low-concentration analytes have focused primarily on assays in which every concentration determination involves consumption of reagents. When numbers of assays become high, due to frequent measurements over long time spans, then reagent consuming approaches are complex and costly. The sensing methodology of the present invention is based on a reversible assay principle, without consuming reagents with each newly recorded concentration datapoint, enabling measurements with high frequency over an endless time span. The described assay principle can be implemented on a variety of sensing platforms, e.g., based on optical, electrical, or acoustical transduction methods, where especially sensing platforms with single-molecule resolution seem suitable since these will allow digital measurements with the highest sensitivity and therefore shortest response times. The described assay principle can be combined with a variety of sampling methods, including remote advection-based sampling and proximal diffusion-based sampling methods. Due to its generalizability and unique and tunable sensing performance, it was found that the limited-volume assay with time-controlled analyte exchange enables studies on time-dependencies of low-concentration analytes and novel applications in the fields of dynamic biological systems, patient monitoring, and biotechnological process control.

### Experimental Section

The experimental section comprises the method of the simulations performed, example calculations and supplementary information referred to throughout the application.

### Finite-Element Analysis.

Finite-element simulations were performed by solving diffusion, advection and reaction equations simultaneously using COMSOL (COMSOL Multiphysics 5.5) and MATLAB (MATLAB R2019a, COMSOL Multiphysics LiveLink for MATLAB) (see Supplementary Information 4). From the simulations, the time-to-equilibrium τ was determined by calculating the time at which the analyte-binder complexes γ_{ab} is at 63% of the difference between the starting level and the equilibrium level of γ_{ab}. The time-controlled analyte exchange (see FIG. 3) was simulated by instantaneously increasing/decreasing the analyte concentration in the measurement chamber Cₐ to C_{a,0}, with which a new measurement cycle starts. The density of analyte-binder complexes γ_{ab}^{start} at the start of a cycle was set to be equal to the density of analyte-binder complexes γ_{ab}^{end} at the end of the preceding cycle. The infinite-volume assay was simulated by forcing the analyte concentration in the measurement chamber Cₐ to be equal to C_{a,0}. Sensor signals are reported at distance L/2 in the measurement chamber (see FIG. 1D). Precisions are reported at a distance L/2 in the measurement chamber, where the signal is collected over a signal collection area of 1 mm² (FIG. 5C-FIG. 5E).

### Fluid Cell Assembly

Glass slides (25×75 mm, #5, Menzel-Glaser) were cleaned by 40 minutes sonication in isopropanol (VWR, absolute) and twice by 10 minutes sonication in MilliQ (ThermoFischer Scientific, Pacific AFT 20). Subsequently, the glass slides were dried under nitrogen flow. A polymer mixture of PLL(20)-g[3.5]-PEG(2) (SuSoS) and PLL(15)-g[3.5]-PEG(2)-N₃ (Nanosoft Polymers) was prepared at a final concentration of 0.45 mg mL⁻¹ and 0.05 mg mL⁻¹ in MilliQ respectively. The glass slides were treated by oxygen plasma (Plasmatreat GmbH) for 1 minute. A custom-made fluid cell sticker (Grace Biolabs), with an approximate volume of 20 μL, was attached to the glass slide and immediately filled with the polymer mixture. After 2 hour incubation, the polymer mixture was removed and the fluid cell was immediately filled with 0.5 nM dsDNA tether solution (221 bp, with DBCO at one end and biotin at the other end) in 0.5 M NaCl in PBS. After overnight incubation, the solution in the fluid cell was exchanged by 2 pM DBCO-functionalized dsDNA solution in 0.5 M NaCl in PBS and incubated for several days until use.

### Particle Functionalization

2 μL streptavidin-functionalized particles (10 mg/mL, Dynabeads MyOne Streptavidin C1, Thermo Fisher Scientific) was incubated with 1 μL biotinylated ssDNA binder molecules (10 μM, IDT, HPLC purification) and 4 μL PBS for 70 minutes. The particles were magnetically washed in 5 vol.-% Tween-20 (Sigma-Aldrich) in PBS and resuspended in 0.5 M NaCl in PBS to a final concentration of 0.1 mg/mL and sonicated using an ultrasonic probe (Hielscher).

### BPM Assay

25 μL particle solution was added to the fluid cell and incubated for 10 minutes. After incubation, the fluid cell was reversed causing unbound particles to sediment. After washing with 40 μL 0.5 M NaCl in PBS, 40 μL mPEG-biotin (500 μM, PG1-BN-1 k, Nanocs) in 0.5 M NaCl in PBS was added to the fluid cell. After 15 minutes incubation, the fluid cell was washed twice with 40 μL PBS. A mixture of ssDNA analytes (IDT, standard desalting) and free binder molecules in PBS was added to the flow cell at the required concentration, immediately after preparation. The sample was observed under a white light source using a microscope (Leica DMI5000M) with a dark field illumination setup at a total magnification of 10× (Leica objective, N plan EPI 10×/0.25 BD). A field of view of approximately 1100×700 μm² was imaged using a CMOS camera (FLIR, Grasshopper3, GS3-U3-23S6M-C) with an integration time of 5 ms and a sampling frequency of 30 Hz. The particles were tracked by applying a phasor-based localization method. The particle activity was determined from the x- and y-trajectories of all particles, by applying a maximum-likelihood multiple-windows change point detection algorithm. The particle activity at equilibrium and the time-to-equilibrium were extracted by fitting the measured particle activity over time using the equation given in Note 3.

### Supplementary Information 1: Standard Parameter Values

Standard parameter values used throughout the application and the Supplementary Information are listed in Table 2.

### Supplementary Information 2: Analytical Expression of the Dose-Response Curve

In a limited-volume sensor with time-controlled analyte exchange, a limited number of analytes interact with binder molecules present in a measurement volume. The input analyte concentration C_{a,0} can be derived from the time-evolution of the density of surface-bound analyte-binder complexes γ_{ab}. It is assumed that all binder molecules are immobilized on a surface, with effective volumetric concentration C_{b,ch}=Γ_{b}/H where Γ_{b} is the density of surface binders and H the height of the measurement chamber. It is assumed that binder molecules are in excess compared to analytes (C_{b,ch}>C_{a,0}) and are in excess compared to the equilibrium dissociation constant (C_{b,ch}>K_{d}). Furthermore, it is assumed that during the secondary exchange phase no analytes are exchanged between the system of interest and the measurement chamber. Assuming first-order Langmuir kinetics, the change in effective volumetric analyte-binder complex concentration per unit time can be determined by:

$$\frac{{dC}_{ab} ( t )}{dt} = {k}_{o n} ( {C}_{a , 0} - {C}_{ab} ( t ) + {f}_{init} {C}_{b , ch} ) {C}_{b , ch} - {k}_{off} {C}_{ab} ( t ) \\ with \\ \frac{{dC}_{ab} ( t )}{dt} \qquad ( Equation S1 )$$

being the time-derivative of the (spatial-dependent) effective volumetric analyte-binder complex concentration Cab, kₒₙ the association rate constant, C_{a,0} the input analyte concentration, fᵢₙᵢₜ the initial fractional occupancy of the binder by an analyte, C_{b,ch} the total effective binder concentration, and k_{off} the dissociation rate constant. Using Cab(t)=γ_{ab}(t)/H, where γ_{ab} is the density of analyte-binder complexes, Equation S1 can be rewritten as a surface reaction rate:

$$\frac{d {γ}_{ab} ( t )}{dt} = {k}_{o n} ( {C}_{a , 0} + {f}_{init} \frac{{Γ}_{b}}{H} ) {Γ}_{b} - {k}_{o n} ( \frac{{Γ}_{b}}{H} + {K}_{d} ) {γ}_{ab} ( t ) with \frac{d {γ}_{ab} ( t )}{dt} \qquad ( Equation S2 )$$

being the time-derivative of the density γ_{ab} of analyte-binder complexes, Γ_{b} the binder density, and K_{d} the equilibrium dissociation constant. To calculate the time-dependent response of the sensor, the differential equation was solved given in Equation S2 in Note 3 and get the general solution for the time-evolution of the density γ_{ab} of analyte-binder complexes after instantaneous analyte exchange and where no mass transport effects are considered.

$$\begin{matrix}Note 3. Derivation of the analytical expression for the time - evolution of \\ the density {γ}_{ab} of analyte - binder complexes after instantaneous analyte \\ exchange\end{matrix}$$

$$\frac{d {γ}_{ab} ( t )}{dt} = {k}_{on} ( {C}_{a , 0} + {f}_{init} \frac{{Γ}_{b}}{H} ) {Γ}_{b} - {k}_{on} ( \frac{{Γ}_{b}}{H} + {K}_{d} ) {γ}_{ab} ( t )$$

$$\frac{d {γ}_{ab} ( t )}{dt} + {C}_{1} {γ}_{ab} ( t ) = {C}_{2} \to {γ}_{ab} ( t ) = \frac{{C}_{2}}{{C}_{1}} + {C}_{3} {e}^{- At} = \frac{{Γ}_{b}}{{Γ}_{b} / H + {K}_{d}} ( {C}_{a , 0} + {f}_{init} \frac{{Γ}_{b}}{H} ) + {C}_{3} {e}^{- {k}_{on} ( {Γ}_{b} / H + {K}_{d} ) t}$$

$${γ}_{ab} ( t = 0 ) = {f}_{init} {Γ}_{b} = \frac{{Γ}_{b}}{{Γ}_{b} / H + {K}_{d}} ( {C}_{a , 0} + {f}_{init} \frac{{Γ}_{b}}{H} ) + {C}_{3} \to {C}_{3} = {f}_{init} {Γ}_{b} - \frac{{Γ}_{b}}{{Γ}_{b} / H + {K}_{d}} ( {C}_{a , 0} + {f}_{init} \frac{{Γ}_{b}}{H} )$$

$${γ}_{ab} ( t ) = \frac{{Γ}_{b}}{{Γ}_{b} / H + {K}_{d}} ( {C}_{a , 0} + {f}_{init} \frac{{Γ}_{b}}{H} ) + ( {f}_{init} {Γ}_{b} - \frac{{Γ}_{b}}{{Γ}_{b} / H + {K}_{d}} ( {C}_{a , 0} + {f}_{init} \frac{{Γ}_{b}}{H} ) ) {e}^{- {k}_{on} ( {Γ}_{b} / H + {K}_{d} ) t}$$

$${γ}_{ab} ( t ) = \frac{α}{α + 1} ( H {C}_{a , 0} + {f}_{init} {Γ}_{b} ) + β {e}^{- t / {τ}_{R}}$$

Here, α=Γ_{b}/(HK_{d}) is the acceleration factor (see Table 2),

$$β = {f}_{init} {Γ}_{b} - \frac{α}{α + 1} ( H {C}_{a , 0} + {f}_{init} {Γ}_{b} ) , and {τ}_{R} = {( {k}_{on} ( \frac{{Γ}_{b}}{H} + {K}_{d} ) )}^{- 1}$$

is the characteristic time-to-equilibrium of the reaction. When the γ_{ab} reaches equilibrium (i.e., t→∞), then

$${γ}_{ab} = {γ}_{ab}^{end} = \frac{α}{α + 1} ( H {C}_{a , 0} + {f}_{init} {Γ}_{b} ) .$$

For a limited-volume sensor with C_{b,ch}>C_{a,0} and C_{b,ch}>K_{d} in the equilibrium condition, nearly all analytes are bound

$$( {f}_{bound} = \frac{α}{α + 1} ≅ 1 )$$

and only a small fraction of analytes is unbound

$$( {f}_{unbound} = \frac{1}{α + 1} ) .$$

Therefore τ_{R} can be simplified to τ_{R}=H/(kₒₙΓ_{b}).

TABLE 2
Standard parameter values
used in the finite-element simulations. Details about
the simulations are described in Supplementary Information 4.

| | Parameter | Value | | Description |
|---|---|---|---|---|
| Input | H | 200 | μm | Measurement |
| | | | | chamber height |
| | L | 1 | cm | Measurement |
| | | | | chamber length |
| | W | 2 | mm | Measurement |
| | | | | chamber width |
| | D | 10⁻¹⁰ | m² s⁻¹ | Diffusion |
| | | | | coefficient |
| | | | | of the analyte |
| | Q | 100 | μL min⁻¹ | Flow rate during |
| | | | | analyte exchange |
| | Γ_{b} | 10⁻⁹ mol m⁻² | | Binder surface |
| | | (600 μm⁻²) | | density |
| | k_{off} | 10⁻⁴ | s⁻¹ | Dissociation rate |
| | | | | constant |
| | kₒₙ | 10⁶ | M⁻¹ s⁻¹ | Association rate |
| | | | | constant |
| | C_{a,0} | 0.1 | pM | Analyte |
| | | | | concentration |
| | Aₛ | 1 | mm² | Signal collection |
| | | | | area |
| Derived | λ = L/H | 50 | | Aspect ratio of |
| | | | | measurement |
| | | | | chamber |
| | τ_{D} = H²/D | 400 | s | Characteristic |
| | | | | diffusion time |
| | τ_{A} = HLW/Q | 2.4 | s | Characteristic |
| | | | | advection time |
| | ${τ}_{R , LV} = \frac{H}{{k}_{on} {Γ}_{b}}$ | 200 | s | Characteristic reaction time for limited-volume assay with C_{b,ch} >> C_{a,0} and C_{b,ch} >> K_{d} |
| | C_{b,ch} = Γ_{b}/H | 5 | nM | Effective |
| | | | | volumetric binder |
| | | | | concentration |
| | K_{d} = k_{off}/kₒₙ | 0.1 | nM | Equilibrium |
| | | | | dissociation |
| | | | | constant |

| | | |
|---|---|---|
| $\text{} \begin{matrix}α = \frac{{Γ}_{b}}{{HK}_{d}} = \\ {C}_{b , ch} / {K}_{d}\end{matrix}$ | 50 | Acceleration factor: reduction factor of the time-to- equilibrium of a limited-volume biosensor (with τ(H, Γ_{b})) compared to an infinite-volume biosensor (with τ(k_{off})). |
| Da = τ_{D}/τ_{R,LV} = | 2 | Damköhler number |
| kₒₙΓ_{b}H/D | | |
| $\text{} \begin{matrix}{Pe}_{L} = \\ \frac{{τ}_{D}}{{τ}_{A}} = \frac{Q}{λ DW}\end{matrix}$ | 167 | Longitudinal Péclet number |
| $\text{} \begin{matrix}{f}_{eq , IV} = \\ \frac{{C}_{a , 0}}{{C}_{a , 0} + {K}_{d}}\end{matrix}$ | 10⁻³ | Equilibrium value of the fractional occupancy in an infinite-volume assay |

In the equation of the time-evolution of the density of analyte-binder complexes (see Note 3), the depletion and repletion regimes can be recognized. When fᵢₙᵢₜ<C_{a,0}/K_{d}, then β<0, so the sensor shows depletion behavior. Conversely, if fᵢₙᵢₜ>C_{a,0}/K_{d}, then β>0 and the sensor shows repletion behavior. Rewriting the time-evolution of the density of analyte-binder complexes using the fractional occupancy f=γ_{ab}/Γ_{b} and t→∞, yields the dose-response relationship as visualized in FIG. 5A:

$${f}_{end} = \frac{α}{α + 1} ( \frac{H}{{Γ}_{b}} {C}_{a , 0} + {f}_{init} ) ≅ \frac{H}{{Γ}_{b}} {C}_{a , 0} + {f}_{init} \qquad ( Equation S3 )$$

Therefore f_{end} depends linearly on C_{a,0}, independent of the value of fᵢₙᵢₜ. The change of fractional occupancy Δf yields the dose-response relation as visualized in FIG. 5B:

$$Δ f = {f}_{end} - {f}_{init} = \frac{α}{α + 1} ( \frac{H}{{Γ}_{b}} {C}_{a , 0} + {f}_{init} ) - {f}_{init} = \frac{1}{α + 1} ( \frac{{C}_{a , 0}}{{K}_{d}} - {f}_{init} ) ≅ \frac{H}{{Γ}_{b}} ( {C}_{a , 0} - {K}_{d} {f}_{init} ) \qquad ( Equation S4 )$$

Here, Δf depends linearly on C_{a,0}, independent of the value of fᵢₙᵢₜ.

### Supplementary Information 3: Sensitivity

The precision of the concentration output of the sensor, is the precision with which the analyte concentration in an unknown sample can be determined using the limited-volume assay. Under the assumption that the measured signal change ΔS=Sᵢₙᵢₜ−S_{end} scales linearly with the change in fractional occupancy Δf, i.e., ΔS∝Δf, the precision of C_{a,0} is calculated using the precision with which Sᵢₙᵢₜ and S_{end} can be determined.

It was assumed that measurement variabilities are dominated by Poisson noise in the number of bound analytes; other factors contributing to variability are not taken into account. An analytical expression is derived for the precision of the analyte concentration C_{a,0} in Note 4 of which the results are given in FIG. 5C-FIG. 5E.

Note 4. Derivation of the analytical expression for the
precision of the analyte concentration C_{a,0} based on Poisson noise only

The error of the signal σ_{S} without background can be estimated by:
${σ}_{{N}_{ab}^{end}} = \sqrt{{N}_{ab}^{end}} and {σ}_{{N}_{ab}^{init}} = \sqrt{{N}_{ab}^{init}}$
where N_{ab}^{end} and N_{ab}^{init} are the total number of analytes bound to binder
molecules that contribute to the signal of the sensor, at the end and start of
a measurement respectively.
The measurement signal change equals ΔS = S_{end} − Sᵢₙᵢₜ. Thus the squared
signal change error is equal to the sum of the squared errors of the two
terms:
${σ}_{Δ S}^{2} = {σ}_{{S}_{end}}^{2} + {σ}_{{S}_{init}}^{2} = {( \frac{{S}_{end}}{\sqrt{{N}_{ab}^{end}}} )}^{2} + {( \frac{{S}_{init}}{\sqrt{{N}_{ab}^{init}}} )}^{2}$
The concentration of the analyte is the output of the sensor. The output
precision can be determined using the signal change error and the slope of
the calibration curve, i.e., the slope of the dose-response curve: σ_{C} =
$\frac{δ {C}_{a , 0}}{δ Δ S} {σ}_{Δ S} with \frac{δ Δ S}{δ {C}_{a , 0}} the concentration ‐ derivative of the signal change .$
This gives the following expression for the error of the concentration
determined using a sensor with Poisson-limited precision:
${σ}_{C} = \frac{δ {C}_{a , 0}}{δΔ S} \sqrt{{( \frac{{S}_{end}}{\sqrt{{N}_{ab}^{end}}} )}^{2} + {( \frac{{S}_{init}}{\sqrt{{N}_{ab}^{init}}} )}^{2}}$

The derivation in Note 4 gives the following analytical expression for the precision of the sensor output, i.e., the error of the concentration cxc:

$${σ}_{C} = \frac{δ {C}_{a , 0}}{δ Δ S} \sqrt{{( \frac{{S}_{end}}{\sqrt{{N}_{ab}^{end}}} )}^{2} + {( \frac{{S}_{init}}{\sqrt{{N}_{ab}^{init}}} )}^{2}} \qquad ( Equation S5 )$$

Equation S5 shows that σ_{C} decreases (i.e., the precision increases) for an increasing number of analytes N_{ab}^{end} for a given signal collection area (see Table 2), for instance by increasing the height of the measurement chamber or the binder density (see Note 3). Since S_{end} and thus N_{ab}^{end} scale linearly with the analyte concentration (see Equation S3), the following can be derived:

$${σ}_{C} ∝ \sqrt{{C}_{a , 0}} \to C {V}_{C} = \frac{{σ}_{C}}{{c}_{a , 0}} ∝ \frac{\sqrt{{C}_{a , 0}}}{{C}_{a , 0}} = \frac{1}{\sqrt{{C}_{a , 0}}} \qquad ( Equation S6 )$$

which results in a 1:2 slope (CV_{C}: C_{a,0}) in FIG. 5A for low fᵢₙᵢₜ. However, if fᵢₙᵢₜ is close to or higher than HC_{a,0}/Γ_{b}, then the contribution of fᵢₙᵢₜ to the fractional occupancy f_{end} at the end of a measurement cycle is relatively large, which results in a rather flat segment in the f_{end}-C_{a,0} curve as visualized in FIG. 5A. Since f_{end}˜fᵢₙᵢₜ, σ_{C} is largely determined by fᵢₙᵢₜ, then Equation S6 converts to

$$C {V}_{c} = \frac{{σ}_{C}}{{C}_{a , 0}} ∝ \frac{1}{{C}_{a , 0}} ,$$

resulting in a 1:1 slope (CV_{C}:C_{a,0}) in FIG. 5C for high fᵢₙᵢₜ.

### Supplementary Information 4: Nondimensionalization

The simulation study of the time-dependent behavior of the biochemical assay was performed using dimensionless parameters for all mass transport processes and reaction rates. The nondimensionalized parameters for mass transport by diffusion and advection are given in Table 3.

TABLE 3
Dimensionless parameters used in the finite-element analysis
for modeling mass transport by diffusion and advection.

| Dimensionless parameter | Symbol | Expression |
|---|---|---|
| Analyte concentration | \tilde{c} | \tilde{c} = Cₐ/C_{a, o} |
| Longitudinal distance | \tilde{x} | \tilde{x} = x/L |
| Transversal distance | \tilde{y} | \tilde{y} = y/H |
| Time | \tilde{t} | t = H²/D → \tilde{t} = t D/H² |

For all finite-element analyses, the time was nondimensionalized using the diffusion time τ_{D} (e.g., FIG. 2B) and thereafter recalculated to normalize with respect to other time scales (e.g., τ_{R} in FIG. 2A and FIG. 2C). When advective flow is included, the used analytical expression of the advective flow is given by:

$$\vec{v} ( y ) = \frac{6 Q}{W {H}^{3}} y ( H - y ) \vec{{e}_{x}} \qquad ( Equation S7 )$$

with \vec{v}(y) the flow speed as a function of the height inside the measurement chamber y, Q the flow rate, W the width of the measurement chamber, and H the height of the measurement chamber. The general equation used in the simulation to describe mass transport by advection and diffusion is given by:

$$\frac{δ {C}_{a}}{δ t} = D {∇}^{2} {C}_{a} - \vec{v} ( y ) \cdot ∇ {C}_{a} \\ with \\ \frac{δ {C}_{a}}{δ t} \qquad ( Equation S8 )$$

being the time-derivative of the (spatial-dependent) analyte concentration Cₐ and D the diffusion coefficient. The dimensionless form of Equation S8 using the defined parameters in Table 3 is derived in Note 5.

$$Note 5. Derivation of the dimensionless advection - diffusion equation$$

$$\frac{δ ( \tilde{c} {C}_{a \text{.0}} )}{δ ( \frac{\tilde{t} {H}^{2}}{D} )} = D \frac{{δ}^{2} ( \tilde{c} {C}_{a \text{.0}} )}{{δ ( \tilde{x} L )}^{2}} + D \frac{{δ}^{2} ( \tilde{c} {C}_{a \text{.0}} )}{{δ ( \tilde{y} L )}^{2}} - \frac{6 Q}{{WH}^{3}} \tilde{y} H ( H - \tilde{y} H ) \frac{δ ( \tilde{c} {C}_{a \text{.0}} )}{δ ( \tilde{x} L )}$$

$$\frac{δ \tilde{c}}{δ \tilde{t}} = \frac{{H}^{2}}{D} \frac{1}{{L}^{2}} D \frac{{δ}^{2} \tilde{c}}{δ {\tilde{x}}^{2}} + \frac{{H}^{2}}{D} \frac{1}{{H}^{2}} D \frac{{δ}^{2} \tilde{c}}{δ {\tilde{y}}^{2}} - \frac{{H}^{2}}{D} \frac{6 Q}{{WH}^{3}} \frac{1}{L} \tilde{y} {H}^{2} ( 1 - \tilde{y} ) \frac{δ \tilde{c}}{δ \tilde{x}}$$

$$\frac{δ \tilde{c}}{δ \tilde{t}} = \frac{{H}^{2}}{{L}^{2}} \frac{{δ}^{2} \tilde{c}}{δ {\tilde{x}}^{2}} + \frac{{δ}^{2} \tilde{c}}{δ {\tilde{y}}^{2}} - \frac{6 QH}{LDW} \tilde{y} ( 1 - \tilde{y} ) \frac{δ \tilde{c}}{δ \tilde{x}}$$

Using the derivation given in Note 5, measurement chamber aspect ratio A=L/H, and longitudinal Péclet number

$$P {e}_{L} = \frac{Q}{λ DW}$$

(see Table 2), the simplified dimensionless advection-diffusion equation is given by:

$$\frac{δ \tilde{c}}{δ \tilde{t}} = \frac{1}{{λ}^{2}} \frac{{δ}^{2} \tilde{c}}{δ {\tilde{x}}^{2}} + \frac{{δ}^{2} \tilde{c}}{δ {\tilde{y}}^{2}} - 6 {Pe}_{L} \tilde{y} ( 1 - \tilde{y} ) \frac{δ \tilde{c}}{δ \tilde{x}} \qquad ( Equation S 9 )$$

The nondimensionalized parameters for the reaction rate are given in Table 4.

TABLE 4
Dimensionless parameters used in the finite-element analysis for
modeling the reaction at the sensor surface.

| Dimensionless parameter | Symbol | Expression |
|---|---|---|
| Analyte concentration at the sensor surface | \tilde{c}* | \tilde{c}* = Cₐ*/C_{a, o} |
| Density of analyte-binder complexes | \tilde{y} | \tilde{y} = y_{ab}/(C_{a, 0}H) |
| Time | \tilde{t} | \tilde{t} = t D/H² |

The general equation used in the simulation to model the reaction at the sensor surface is given by:

$$\frac{δ {γ}_{a b} ( t )}{δ t} = {k}_{o n} {C}_{a}^{*} ( {Γ}_{b} - {γ}_{a b} ( t ) ) - {k}_{off} {γ}_{a b} ( t ) with \frac{δ {γ}_{a b}}{δ t} \qquad ( Equation S 11 )$$

the time-derivative of the (spatial-dependent) density γ_{ab} of analyte-binder complexes and Cₐ* the analyte concentration at the sensor surface, which is known by solving Equation S9. The dimensionless form of Equation S11 is derived in Note 6.

$$Note 6. Derivation of the dimensionless reactive rate equation$$

$$\frac{δ ( \tilde{γ} {C}_{a , o} H )}{δ ( \frac{\tilde{t} {H}^{2}}{D} )} = {k}_{o n} {\tilde{c}}^{*} {C}_{a , 0} ( {Γ}_{b} - \tilde{γ} {C}_{a , o} H ) - {k}_{off} \tilde{γ} {C}_{a , o} H$$

$$\frac{δ \tilde{γ}}{δ \tilde{t}} = \frac{{H}^{2}}{D} \frac{{k}_{o n} {\tilde{c}}^{*}}{H} ( {Γ}_{b} - \tilde{γ} {C}_{a , o} H ) - \frac{{H}^{2}}{D} {k}_{off} \tilde{γ}$$

$$\frac{δ \tilde{γ}}{δ \tilde{t}} = \frac{{k}_{o n} {Γ}_{b} H}{D} {\tilde{c}}^{*} - \frac{{k}_{o n} H}{D} {\tilde{c}}^{*} \tilde{γ} {C}_{a , o} H - \frac{{H}^{2}}{D} {k}_{off} \tilde{γ}$$

$$\frac{δ \tilde{γ}}{δ \tilde{t}} = \frac{{C}_{a , o} H}{{Γ}_{b}} \frac{{k}_{o n} {Γ}_{b} H}{D} [ {\tilde{c}}^{*} ( \frac{{Γ}_{b}}{{C}_{a , o} H} - \tilde{γ} ) - \frac{{K}_{d}}{{C}_{a , o}} \tilde{γ} ]$$

Using the derivation given in Note 6 and Damköhler number

$$D a = \frac{{k}_{o n} {Γ}_{b} H}{D}$$

(see Table 2), the simplified dimensionless reactive rate equation is given by:

$$\frac{δ \tilde{γ}}{δ \tilde{t}} = \frac{{C}_{a , o} H}{{Γ}_{b}} Da [ {\tilde{c}}^{*} ( \frac{{Γ}_{b}}{{C}_{a , o} H} - \tilde{γ} ) - \frac{{K}_{d}}{{C}_{a , o}} \tilde{γ} ] \qquad ( Equation S 12 )$$

### Supplementary Information 5: Time-Controlled Analyte Exchange

Time-controlled analyte exchange in a limited-volume assay refers to the switching between the primary exchange phase and the secondary exchange phase (see FIG. 1D). In the primary exchange phase, analytes are exchanged effectively between the system of interest and the measurement chamber, e.g., by diffusion and/or advection. In the secondary exchange phase, analytes are exchanged ineffectively between the system of interest and the measurement chamber, causing a limited-volume incubation in the measurement chamber. The analyte exchange can be controlled in time by changing the mass transport between the system of interest and the measurement chamber, e.g., by stopping flow and/or diffusion. It is assumed that there is no analyte exchange during the secondary exchange phase (i.e., τ_{sec.exch}→∞) causing time scale τ_{sec.exch} and duration t_{sec.exch} to be irrelevant; therefore the characteristic time of primary exchange is indicated by τ_{exch} and the duration of the primary exchange is indicated by t_{exch}, both without the primary subscript.

FIG. 6 shows how time-controlled analyte exchange influences the performance of the sensor, for two analyte exchange principles, namely remote advection-based sampling and proximal diffusion-based sampling. In Supplementary Information 6, the influence of time-controlled analyte exchange on the performance of the sensor is quantified by simulating the time-to-equilibrium τ and the coefficient of variation of the concentration CV_{c} as a function of the duration of the primary exchange phase t_{exch}.

FIG. 6A schematically visualizes time-controlled analyte exchange by advection (top) and by diffusion (bottom), where the primary exchange has a characteristic time τ_{exch} and a duration t_{exch} (see FIG. 6B). t_{exch} is controlled by controlling the flow rate Q or the molar flux Jₐ (by controlling the membrane permeability P) in time.³ For advection-based sampling, τ_{exch} equals the characteristic advection time τ_{A} (see Table 2), while for diffusion-based sampling, τ_{exch} equals the characteristic diffusion time τ_{D} (see Table 2). In t_{exch}, analytes are transported over a characteristic length L_{A}=t_{exch}Q/(HW) by advection and L_{D}=\sqrt{t_{exch}D} by diffusion.

Three regimes can be identified with regard to the analyte exchange. First, t_{exch}<τ_{exch} implies that LA or LD is shorter than the length L of the measurement chamber or the height H of the measurement chamber for advection-based sampling and diffusion-based sampling respectively. Second, If t_{exch} equals τ_{exch}, the transport distance equals L or H for advection-based sampling and diffusion-based sampling respectively; this condition is used in FIG. 2C for analyte exchange by advection, since here the exchanged volume equals the volume of the measurement chamber. Third, t_{exch}>τ_{exch} implies that L_{A} or L_{D} is longer than L or H for advection-based sampling and diffusion-based sampling respectively.

In the simulations the following assumptions were made. First, analyte exchange between the system of interest and the measurement chamber only occurs during the primary exchange phase. For advection-based analyte exchange, this implies that the flow rate Q is high in phase 1 (the primary exchange phase) and zero in phase 2 (the secondary exchange phase). For diffusion-based analyte exchange, the membrane permeability P is high in phase 1, and zero in phase 2. The second assumption is that diffusive mass transport within the measurement chamber itself occurs at all times.

Analyte exchange can be controlled by controlling the characteristic analyte exchange time τ_{exch} (by design parameters Q and P) and by controlling the analyte exchange duration t_{exch}. In Supplementary Information 6 the performance as a function of Q and t_{exch} for advection-based exchange is studied. For diffusion-based exchange, the performance as a function of t_{exch} is studied, assuming P=0 or P→∞.

The ratio between mass transport facilitated by diffusion versus mass transport facilitated by advection can be compared using the longitudinal Peclet number Pe_{L}:

$$P {e}_{L} = \frac{{k}_{A}}{{k}_{D}} = \frac{{τ}_{D}}{{τ}_{A}} = \frac{{H}^{2}}{D} \frac{Q}{H L W} = \frac{Q}{λ DW} \qquad ( Equation S 13 )$$

with k_{A} being the mass transport rate due to advection (here over distance L), kD the mass transport rate due to diffusion (here over distance H), τ_{D} the characteristic diffusive time scale, τ_{A} the characteristic advective time scale, H the height of the measurement chamber, D the diffusion coefficient of an analyte, Q the flow rate, W the width of the measurement chamber, and λ=L/H the aspect ratio of the measurement chamber. From Equation S13 can be concluded that for Pe_{L}>1 the τ_{D} is larger than τ_{A} and therefore the mass transport is diffusion-limited. For Pe_{L}<1, τ_{D} is smaller than τ_{A} which causes the mass transport to be advection-limited.

### Supplementary Information 6: The Influence of Time-Controlled Analyte Exchange on the Sensor Performance

The influence of flow rate Q on the observed time-to-equilibrium τ and the precision of the concentration determination CV_{C} is quantified in FIG. 7 for a sensor with standard parameter values as listed in Table 2 and a geometry as given in FIG. 6A, top. FIG. 7A shows the time-to-equilibrium τ as a function of longitudinal Peclet number Pe_{L}, by varying the flow rate Q, for three values of t_{exch}/τ_{A} (see Supplementary Information 5). For small Pe_{L} the mass transport by advection is slow compared to mass transport by diffusion, and thus τ is advection-limited and scales according to τ∝1/Q (see Note 7). Besides, increasing the duration of the primary exchange t_{exch} causes the observed time-to-equilibrium τ to be longer. The plateau value (dashed line) is reached when mass transport by advection is fast compared to mass transport by diffusion, i.e., at high flow rates, which corresponds to the τ found in FIG. 2 for an equal sensor height. In this regime, the sensor can be assumed to be incubated with a concentration C_{a,0} instantaneously. For Pe_{L}→∞, a τ is observed which equals the found τ for instantaneous analyte exchange (see FIG. 2A-FIG. 2B).

$$Note 7. The influence of analyte exchange by advection on the \text{time-to-equilibrium} τ$$

$$When the \text{time -to-equilibrium} is \text{advection-limited} , this results in τ = {t}_{e x c h} :$$

$$τ = {t}_{e x c h} \to τ = \frac{HLW}{Q} \frac{{t}_{e x c h}}{{τ}_{A}} = \frac{λ DW}{Q} \frac{HL}{λ D} \frac{{t}_{e x c h}}{{τ}_{A}} = \frac{1}{P {e}_{L}} \frac{{H}^{2}}{D} \frac{{t}_{e x c h}}{{τ}_{A}}$$

$$τ ∝ \frac{1}{P {e}_{L}} \to τ : P {e}_{L} = 1 : 1 .$$

FIG. 7B shows the precision of the concentration determination as a function of the longitudinal Peclet number Pe_{L} and flow rate Q on the secondary x-axis. Again, roughly two regimes can be identified: for small Pe_{L}, CV_{C} depends on Pe_{L} since the analytes entering the measurement chamber bind to the binders on surface, which results in longitudinal depletion; this results in a positional dependency of the density γ_{ab} of analyte-binder complexes and thus the precision. For large Pe_{L}, the analyte exchange by advection is much faster than analyte exchange by diffusion causing almost instantaneous exchange of material, which results in lateral depletion, and thus an independency of the precision on Pe_{L}. For Pe_{L}→∞, a CV_{C} is observed which equals the found CV_{C} for instantaneous analyte exchange (see FIG. 5D-FIG. 5E).

FIG. 8 shows the influence of diffusion-based analyte exchange on the observed time-to-equilibrium, by comparing instantaneous analyte exchange (see FIG. 2) to diffusion-based analyte exchange, where t_{exch}/τ_{D}=1. FIG. 8A shows the time-to-equilibrium τ as a function of Damköhler number Da for diffusion-based analyte exchange (dark orange) and instantaneous analyte exchange (light orange, same data as FIG. 2A), by varying the height H of the measurement chamber. At low Da (i.e., at small measurement chamber height H), for both exchange methods, the observed time-to-equilibrium is reaction-limited since the diffusion time scale is fast compared to the reaction time scale. However, for increasing Da, the time-to-equilibrium increases faster for diffusion-based exchange since the characteristic length scale L_{D} over which the molecules have to diffuse is larger (L_{D}=H) compared to instantaneous analyte exchange (on average L_{D}≅H/2).

FIG. 8B shows the time-to-equilibrium τ as a function of Da for diffusion-based analyte exchange (dark orange) and instantaneous analyte exchange (light orange, same data as FIG. 2B), by varying the binder density Γ_{b}. At low Da (i.e., at low surface binder density Γ_{b}), for both exchange methods, the observed time-to-equilibrium is reaction-limited since the reaction time scale is slow (τ_{R}=1/k_{off}) compared to the diffusion time scale. However, at high Da where the time-to-equilibrium τ is determined by the diffusion time scale, τ is larger for diffusion-based exchange since the characteristic length scale L_{D} over which the molecules have to diffuse is larger.

FIG. 9 shows the influence of the primary exchange on the time-to-equilibrium and the sensitivity of the sensor for two monitoring geometries as presented in FIG. 6 and using the standard parameter values given in Table 2. FIG. 9A sketches the time-evolution of the fractional occupancy (solid orange line) and the primary exchange (dashed light orange line) by advection (by controlling flow rate Q) or diffusion (by controlling membrane permeability P). Three regimes are given here, where (1) τ is determined by the mass transport by diffusion within the measurement chamber, (2) τ is determined by the duration of the primary exchange t_{exch}, and (3) τ is determined by the dissociation rate constant k_{off}. Note that for a short t_{exch}, the fractional occupancy when equilibrium is reached, is lower, since less molecules are exchanged between the measurement chamber and the system of interest.

FIG. 9B shows the simulated results of the time-to-equilibrium τ normalized to the diffusion time scale τ_{D} as a function of the duration of the primary exchange t_{exch} normalized to the diffusion time scale τ_{D} (left) and the coefficient of variation of the concentration CV_{C} as a function of t_{exch}/τ_{D} (right). The three regimes as visualized in panel a could be observed in both graphs. For a small t_{exch}/τ_{D}, the observed time-to-equilibrium τ is diffusion-limited, since t_{exch} is much smaller than τ_{D}. However, CV_{C} is high (i.e., precision is low) since the number of exchanged analytes is small; here, CV_{C} scales according to

$$C {V}_{C} ∝ 1 / \sqrt[4]{{t}_{e x c h} / {τ}_{D}}$$

(see Note 8). For a large t_{exch}/τ_{D}, the observed time-to-equilibrium τ is reaction-limited, since the assay converts into an infinite-volume assay. However, CV_{C} is low (i.e., precision is high) since the number of exchanged analytes is large (i.e., there is an infinite supply of analytes). Here the precision is independent of the primary exchange, since the reaction reaches an equilibrium under infinite supply of analytes. When τ˜t_{exch}, the time-to-equilibrium is mainly determined by the duration of the primary exchange since the assay can be regarded as neither a limited-volume assay nor an infinite-volume assay; here, CV_{C} scales roughly according to CV_{C}∞1/\sqrt{t_{exch}/τ_{D}} (see Note 8).

Note 8. The mathematical dependency of
sensitivity for analyte exchange by diffusion

Fick's First Law gives Jₐ^{D} = −D∇Cₐ, where Jₐ^{D} is the diffusion flux of
$analytes , which translates into {J}_{a}^{D} = - D \frac{δ {C}_{a}}{δ y} assuming one ‐ dimensional$
transversal diffusion. The maximum density γ_{ab}^{max} of analyte-binder
complexes that can be reached, is calculated by γ_{ab}^{max} = Jₐ^{D}t_{exch}. The
mean length L_{D} over which analytes diffuse during t_{exch} equals δy =
${L}_{D} = \sqrt{{H}^{2} \frac{{t}_{exch}}{{τ}_{D}}} for {L}_{D} < H and δ y = H for {L}_{D} \geq H .$
$For {L}_{D} < H , gives {γ}_{ab}^{max} = {HC}_{a} \sqrt{\frac{{t}_{exch}}{{τ}_{D}}} and for {L}_{D} \geq H , gives {γ}_{a b}^{max} =$
${HC}_{a} \frac{{t}_{exch}}{{τ}_{D}} . The maximum achievable coefficient of variation can be$
$calculated by {CV}_{C} = \frac{1}{\sqrt{{γ}_{ab}^{max} {A}_{S}}} , where {A}_{s} is the signal collection area .$
$For {L}_{D} < H , gives {CV}_{C} ∝ \frac{1}{\sqrt[4]{\frac{{t}_{exch}}{{τ}_{D}}}} and for {L}_{D} \geq H and {CV}_{C} ∝ \frac{1}{\sqrt{\frac{{t}_{exch}}{{τ}_{D}}}} .$

FIG. 9C shows the simulated results of the time-to-equilibrium τ, normalized to the advection time scale τ_{A}, as a function of the duration of the primary exchange t_{exch}, normalized to the advection time scale τ_{A} (left), and the coefficient of variation of the concentration CV_{C} as a function of t_{exch}/τ_{A} (right). Again, the three regimes as visualized in panel a are observed in both graphs. The behavior is similar to panel b: for a small t_{exch}/τ_{A}, the observed time-to-equilibrium τ is diffusion-limited, but CV_{C} is high (i.e., precision is low) since the number of exchanged analytes is small. No values are shown for t_{exch}/τ_{A}<1 due to the development of positional dependency of γ_{ab} when the volume of the measurement chamber is not fully exchanged (see FIG. 7B). Besides, at low t_{exch}/τ_{A}, the precision is roughly independent of t_{exch}/τ_{A} due to the development of a lateral depletion zone (see FIG. 7B). For a large t_{exch}/τ_{A}, the observed time-to-equilibrium τ is reaction-limited and CV_{C} is low (i.e., precision is high) since the number of exchanged analytes is large (i.e., there is an infinite supply of analytes). When ˜t_{exch}, the time-to-equilibrium is mainly determined by the duration of the primary exchange and CV_{C} scales roughly according to CV_{C}∝1/t_{exch}/τ_{A} (see Note 9). However this expected CV_{C} based on the number of analytes entering the measurement chamber (see black dashed line, left bottom corner), is lower than the observed CV_{C} (see also panel b). The difference between analyte exchange by diffusion and by advection, is that in the case of analyte exchange by advection, analytes can be lost through the oulet without contributing to the precision of the sensor. Therefore the maximum density γ_{ab}^{max} can be reached in diffusion-based analyte exchange, while it cannot be reached in advection-based analyte exchange.

Note 9. The mathematical dependency
of sensitivity for analyte exchange by advection

The advective flux of analytes Jₐ^{A} can be described by Jₐ^{A} = QC_{a,0}. The
maximum analyte-binder surface density γ_{ab}^{max} can be calculated by
${γ}_{ab}^{max} = \frac{{J}_{a}^{A}}{WL} {t}_{exch} \to {γ}_{ab}^{max} = {HC}_{a , 0} \frac{{t}_{exch}}{{τ}_{A}} , assuming {L}_{A} \geq L with lateral$
depletion. The maximum achievable coefficient of variation can be
$calculated by {CV}_{C} = \frac{1}{\sqrt{{γ}_{a b}^{max} {A}_{s}}} , where {A}_{s} is the signal collection area .$
$For {L}_{A} \geq L , gives {CV}_{C} ∝ \frac{1}{\sqrt{\frac{{t}_{exch}}{{τ}_{A}}}} .$
$\to {CV}_{C} : \frac{{t}_{exch}}{{τ}_{A}} = 1 : 2 for {l}_{adv} \geq L$

### Supplementary Information 7: Biosensing by Particle Mobility

Here the concept of rapid monitoring of low-concentration analytes by time-controlled analyte exchange is experimentally demonstrated using Biosensing by Particle Mobility (BPM), a biosensing method with both single-particle and single-molecule resolution. The molecular design and measurement principle are sketched in FIG. 10, illustrated with a sandwich assay format. FIG. 10A shows a particle that is tethered to a substrate by a dsDNA tether and functionalized with ssDNA binder molecules, and a surface that is functionalized with secondary binder molecules.

FIG. 10B illustrates the sensing functionality of the BPM system. The secondary binder molecules can transiently bind to analytes captured from solution by the binder molecules on the particle. The transient binding affects the mobility of the particle, because an unbound particle has a larger in-plane motional freedom than a bound particle. Two mobility time traces are sketched in FIG. 10C, at a high (left) and low (right) analyte concentration. The switching frequency, i.e., the activity, of the particle depends on the analyte concentration, because the unbound state lifetime of a particle decreases when the number of captured analytes increases.

In order to demonstrate the rapid monitoring methodology for low-concentration analytes using BPM, ssDNA analytes were used that bind with a 20nt interaction to the ssDNA binder molecules on the particle. The particles are functionalized with a high binder density and have a high-affinity interaction with the analyte (characteristic lifetime of several hours), which implies that C_{b,ch}>C_{a,0} and C_{b,ch}>K_{d}, and therefore the effective volumetric binder concentration dominates the time-to-equilibrium of the reaction.

Supplementary Information 8: Precision of Biosensing by Particle Mobility with Time-Controlled Analyte Exchange

The results of BPM measurements with time-controlled analyte exchange are given in FIG. 11. FIG. 11A shows the measured activity per measurement block of 5 minutes as a function of time for multiple consecutive measurement cycles (bottom). At the start of each cycle (see vertical grey lines), the measurement chamber was filled with a solution containing input analyte concentration C_{a,0}=200 μM (middle) and a varying supplemented binder concentration C_{b,suppl} (top). The data show that the time-to-equilibrium is shorter in a condition with high supplemented binder concentration. The data in FIG. 11A were fitted in order to extract values for the time-to-equilibrium τ and for the signal change ΔS; the fitted values for τ and ΔS are plotted in FIG. 4B and discussed in the body text of the description of the present invention.

It is questioned to what extent the precision of the BPM sensor is limited by Poisson statistics (cf. FIG. 5). The total variation observed in a measurement is quantified and the variation induced by the measurement itself is calculated. Using this approach the variation caused by other sources than the measurement can be estimated and compared to the variation caused by the discrete number of analyte-binder complexes on the particles, i.e., the Poisson-limited variation.

FIG. 11B shows a zoom-in of the measurement cycle where C_{b,suppl}=10 nM (top) and the distribution of the observed activity when the reaction is in equilibrium (bottom). The activity as a function of time is shown with a moving average, with a time window T, of 1 s, of which the observed variation of the activity equals σ_{obs}=3 mHz. The activity as a function of time is fitted by a single exponential of the form given in Note 3 (top, dashed line), from which the time-to-equilibrium τ and ΔS were extracted (see FIG. 4B). The distribution of the observed activity is fitted with a normal distribution from which the mean activity μ_{A} and σ_{obs} are extracted.

FIG. 11C shows σ_{obs} as a function of T, for the first cycle (C_{b,suppl}=50 nM, brown) and the second cycle (C_{b,suppl}=10 nM, orange). Increasing the time window TW, results in a smaller σ_{obs} since the calculated activity is averaged over more time points. The observed variation in the activity depends on the variation induced by the measurement and by other sources of variation:

$${σ}_{o b s}^{2} = {σ}_{m e a s}^{2} + {σ}_{o t h e r}^{2} \qquad ( Equation S 14 )$$

where σₘₑₐₛ=σ_{ref}/\sqrt{τ_{w}}, with σₘₑₐₛ the measurement induced variation, σ_{ref} a reference variation which is taken as the measurement induced variation at T_{W}=1 s, T_{W} the time window of the moving average of the activity as a function of time, and σₒₜₕₑᵣ the variation from a different source than the measurement itself, e.g., a discrete number of analyte-binder complexes or variations in surface chemistry. If the precision of a sensor is Poisson-limited, σₒₜₕₑᵣ equals σ_{Poisson}, where σ_{Poisson} is the variation caused by the discrete number of observed analyte-binder complexes within the signal collection area.

For a BPM measurement, the number of observed analyte-binder complexes can be calculated by:

$${N}_{a b}^{o b s} = {γ}_{a b}^{eff} {A}_{S} {ϵ}_{p}^{o b s} \qquad ( Equation S 15 )$$

where γ_{ab}^{eff} is the effective analyte-binder complex density, Aₛ the signal collection area and ϵₚ^{obs} the observed fraction of the particle area. In a BPM measurement with 1 μm particles, only approximately 2% of the particle surface is contributes to the observed signal¹, which results in ϵₚ^{obs}=0.02. γ_{ab}^{eff} can be calculated by:

$${γ}_{a b}^{eff} = {f}_{a}^{eff} {f}_{e n d} {Γ}_{b} \qquad ( Equation S 16 )$$

where fₐ^{eff}=Γ_{b}/(HC_{b,suppl}+Γ_{b}) is the effective fraction of the total analytes captured by the binders on the surface, and f_{end} the fractional occupancy of all binder molecules by analytes at the end of a measurement cycle for a given input analyte concentration C_{a,0}. Note that fₐ^{eff} can be larger than 1 when multiple consecutive cycles have been measured: for the first cycle, fₐ^{eff}=Γ_{b}/(HC_{b,suppl,1}+Γ_{b}), while for the second cycle, fₐ^{eff}=Γ_{b}/(HC_{b,suppl,1}+Γ_{b})+Γ_{b}/(HC_{b,suppl,2}+Γ_{b}). Using standard parameter values from Table 2, f_{end}=10⁻² (extrapolated from FIG. 5A at C_{a,0}=200 pM), and a signal collection area of ΔS=1 mm², it can be found that σ*_{Poisson}=\sqrt{N_{ab}^{obs}}=1.0·10² for the first cycle (where C_{b,suppl}=50 nM) and that σ*_{Poisson}=2.3·10² for the second cycle (where C_{b,suppl}=10 nM). Assuming a Poisson-limited sensor, the variation in the observed activity equals σ_{Poisson}=CV_{Poisson}μ_{A} where CV_{Poisson}=σ*_{Poisson}/N_{ab}^{obs} is the coefficient of variation in the observed number of analyte-binder complexes and μ_{A} the mean observed activity at equilibrium (see panel a). This gives CV_{Poisson}=9.6·10⁻³ and therefore σ_{Poisson}=0.18 mHz for the first incubation cycle (where C_{b,suppl}=50 nM), and CV_{Poisson}=4.4-10⁻³ and therefore σ_{Poisson}=0.18 mHz for the second incubation cycle (where C_{b,suppl}=10 nM).

The dashed lines in FIG. 11C represents the fit according to Equation S14. By taking the limit T_{w}→∞, σ_{obs} equals σₒₜₕₑᵣ. At the first cycle with C_{b,suppl}=50 nM, σₒₜₕₑᵣ was found to be equal to σₒₜₕₑᵣ=0.09+0.02 mHz, while at the second cycle with C_{b,suppl}=10 nM, σₒₜₕₑᵣ=0.21+0.03 mHz. Comparing these values to the previously quantified 6Poisson, one can conclude that σₒₜₕₑᵣ≅σ_{Poisson}, which indicates that the precision in the BPM measurement is Poisson-limited. Therefore, the precision of the BPM measurements are determined by the fundamental limit of stochastic fluctuations in the number of analyte-binder complexes, and can be compared to the results given in FIG. 5C-FIG. 5D.

### BRIEF DESCRIPTION OF THE DRAWINGS

The following section briefly discusses the different drawings.

FIG. 1: concept of the sensing methodology for the rapid monitoring of low analyte concentrations

FIG. 1A. Sensing system for analyte monitoring. Analytes are exchanged between a biological or biotechnological system of interest and a measurement chamber. The data result in a concentration-time profile which should correspond as close as possible to the true analyte concentration in the system of interest. FIG. 1B. Geometry of the measurement chamber, with height H, width W, and length L. A reaction rate at the sensor surface is caused by the association and dissociation between analytes (orange) and binder molecules (brown), described by the association rate constant kₒₙ, the dissociation rate constant k_{off}, the total binder density Γ_{b}, the analyte concentration Cₐ near the surface, and the density of analyte-binder complexes γ_{ab}. Analyte exchange is facilitated by diffusion and advection, where diffusion occurs in both x- and y-direction with diffusion coefficient D, resulting in a net molar flux Jₐ, and where advection occurs in the x-direction only, with a developed flow profile with flow rate Q and a mean flow speed vₘ. FIG. 1C. The time profile of the sensor response for low analyte concentration (C_{a,0}<K_{d}), for two conditions: infinite-volume and limited-volume assays. Measuring in an infinite volume results in an excess of analytes compared to binder molecules (C_{a,0}>C_{b,ch}), causing the time-to-equilibrium τ to be determined by k_{off}. The limited-volume condition is defined as a condition where binder molecules are in excess compared to analytes (C_{b,ch}>C_{a,0}, with C_{b,ch}=Γ_{b}/H) and in excess compared to the equilibrium dissociation constant (C_{b,ch}>K_{d}). This causes τ to be determined by the effective binder concentration (i.e., measurement chamber height H and binder density Γ_{b}), which is much shorter than 1/k_{off}. FIG. 1D. Analyte monitoring using a limited-volume assay involves repeated cycles with two phases. In phase 1, the primary exchange phase, analytes are exchanged effectively between the system of interest and the measurement chamber. In phase 2, the secondary exchange phase, analytes are exchanged ineffectively. Recording the time-dependent signal during the secondary exchange phase (in the middle of the measurement chamber at distance L/2 from the entrance), reveals the analyte concentration. Inside the measurement chamber, the limited-volume condition gives a time-dependence of the analyte concentration: a decrease over time (depletion) or an increase over time (repletion), depending on the input analyte concentration C_{a,0} and the initial occupation fᵢₙᵢₜ of binders by analytes. The input analyte concentration C_{a,0} is derived from the measured time-dependence of the fractional occupation f(t).

FIG. 2: time-to-equilibrium τ of a limited-volume assay for a sensor design with different heights, binder densities, and flow rates of analyte exchange

FIG. 2A. Time-to-equilibrium τ as a function of measurement chamber height H (orange line) for an instantaneous analyte exchange. For small H, the observed τ is reaction-dominated (τ=τ_{R}=1/(τ_{R,LV}⁻¹+k_{off}), black dotted line), while for increasing H the observed τ becomes diffusion-dominated. The inset shows the same data, where τ is normalized to τ_{R} and plotted as a function of Damköhler number Da. The sketch above the graph visualizes a measurement chamber with an increasing measurement chamber height. FIG. 2B. Time-to-equilibrium τ as a function of the binder density Γ_{b} (orange line) for an instantaneous analyte exchange. For low Γ_{b}, the observed τ is reaction-dominated (τ=τ_{R}, black dotted line), while for increasing Γ_{b} the observed τ becomes diffusion-dominated. The inset shows the same data, where τ is normalized to the characteristic diffusion time τ_{D} and plotted as a function of Da. For low Da, τ is limited by 1/k_{off}, while at high Da, τ is limited by τ_{D}. The sketch above the graph visualizes a measurement chamber with an increasing binder density. FIG. 2C. Time-to-equilibrium τ as a function of flow rate Q for three aspect ratios λ=L/H, for time-controlled analyte exchange by advection where the primary exchange phase duration t_{pr.exch} equals the characteristic advection time τ_{A}. For small Q, the observed τ is limited by the advective transport of analytes from the inlet toward the point of sensing at distance L/2 from the inlet. For increasing Q, this transport process becomes faster causing the observed τ to be dominated by reaction and/or diffusion at high flow rates. The inset shows the same data (Da=2) supplemented with Da=0.2 (reaction-limited) and Da=20 (diffusion-limited), where τ is normalized to τ_{R} and plotted as a function of the longitudinal Peclet number Pe_{L}. The dotted lines show the τ/τ_{R} value at high Q and are comparable to the values found in panel a. The sketch above the graph visualizes a measurement chamber with an increasing flow rate. In all panels, the black arrows on the x-axis indicate the standard parameter values for H, Γ_{b}, and Q as listed in Table 1.

FIG. 3: Simulated response of the analyte monitoring system using time-controlled analyte exchange

FIG. 3A. The analyte concentration Cₐ in the measurement chamber (brown line) and the fractional occupancy f of binder molecules by analytes (orange line) as a function of time, for low fᵢₙᵢₜ and depletion of analyte in solution (left), and for high fᵢₙᵢₜ and repletion of analyte in solution (right). The dashed lines indicate time points where instantaneous analyte exchange occurs, where the bulk analyte concentration was set to C_{a,0}=0.1 pM after a period of approximately 50 min. The insets highlight the kinetics of the first cycles, showing a time-to-equilibrium of τ=340 s. For many cycles (n→∞) both curves would approach

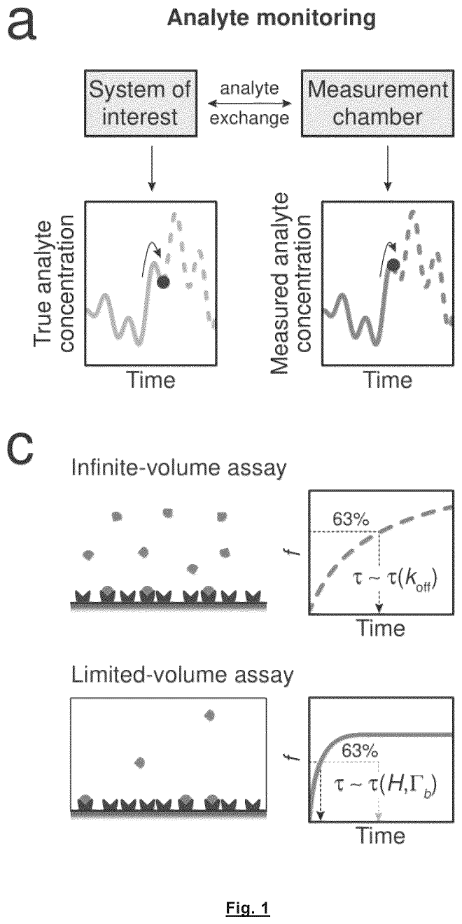

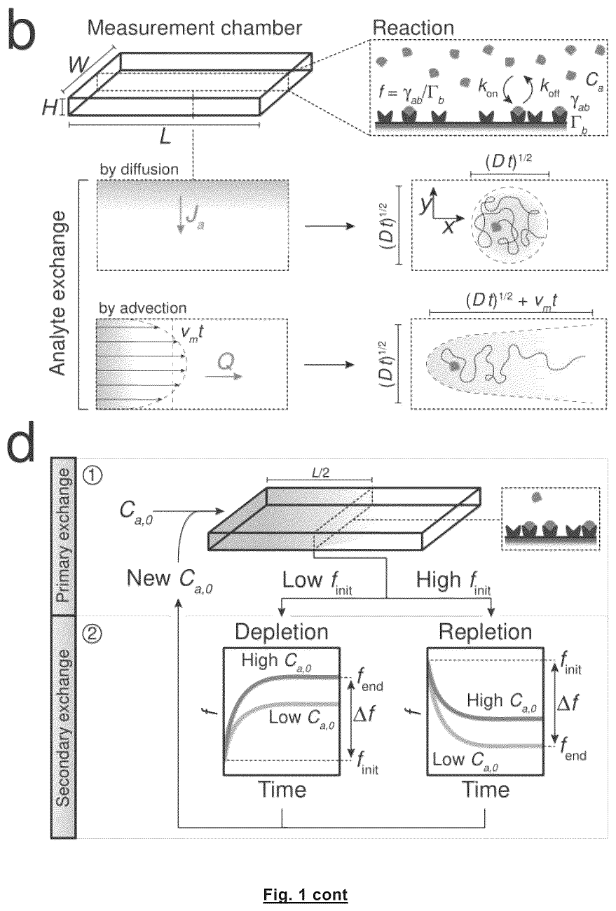

$${f}_{eq , IV} = \frac{{C}_{a , 0}}{{C}_{a , 0} + {K}_{d}} = 10 \cdot {10}^{- 4} ,$$

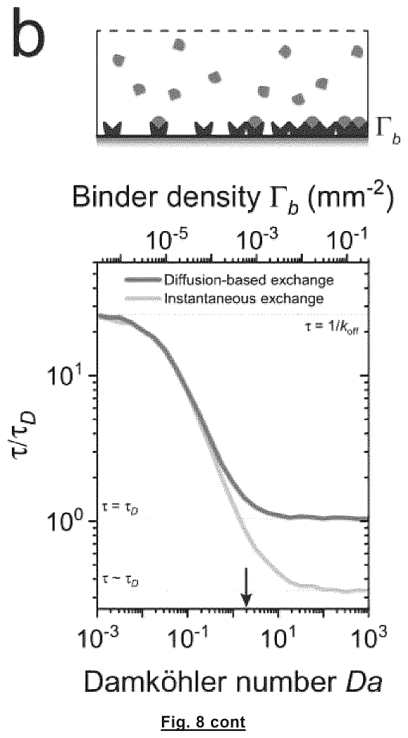

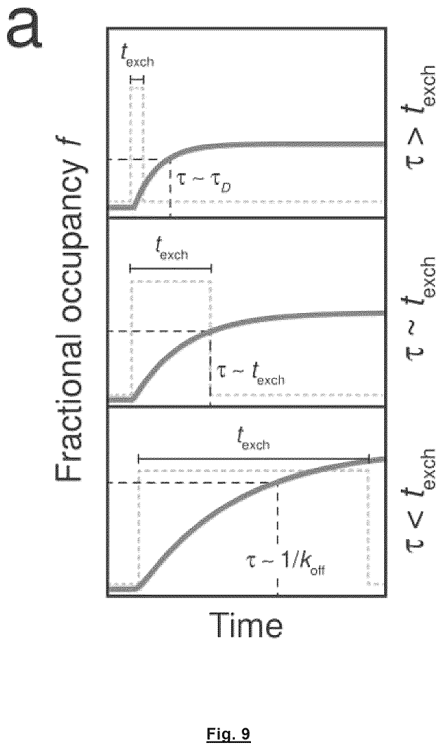

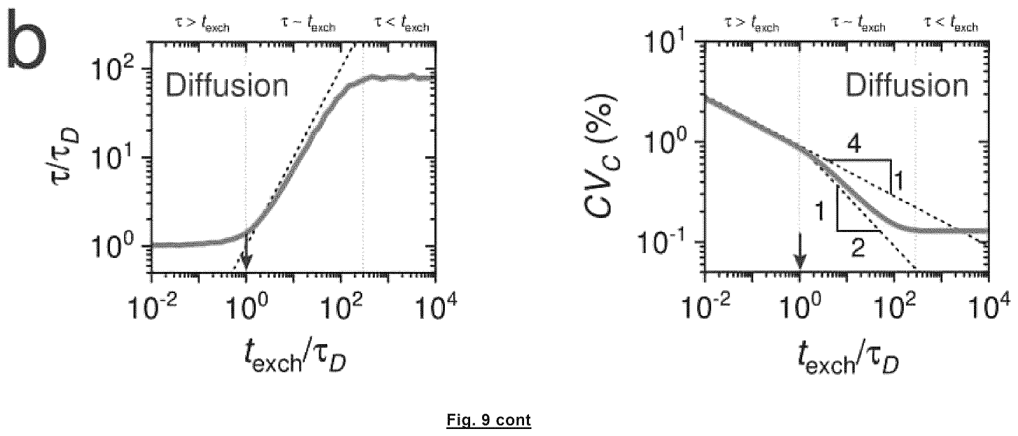

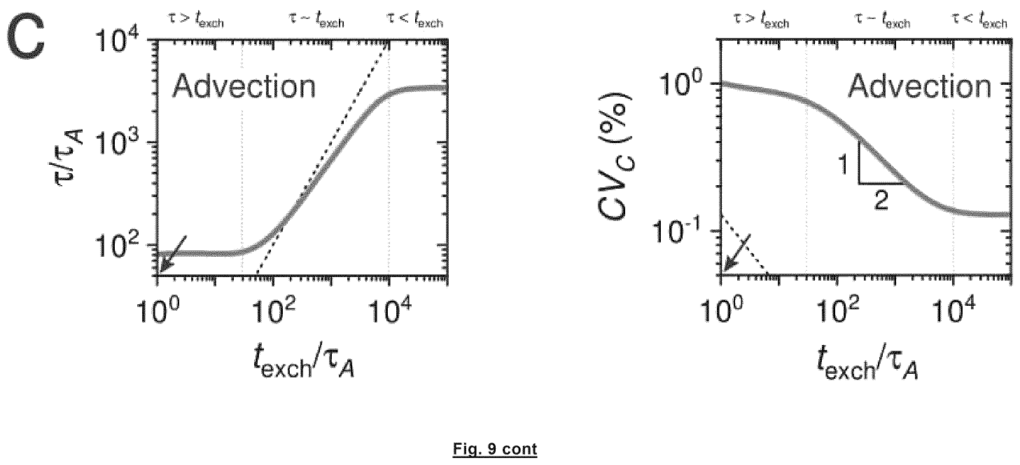

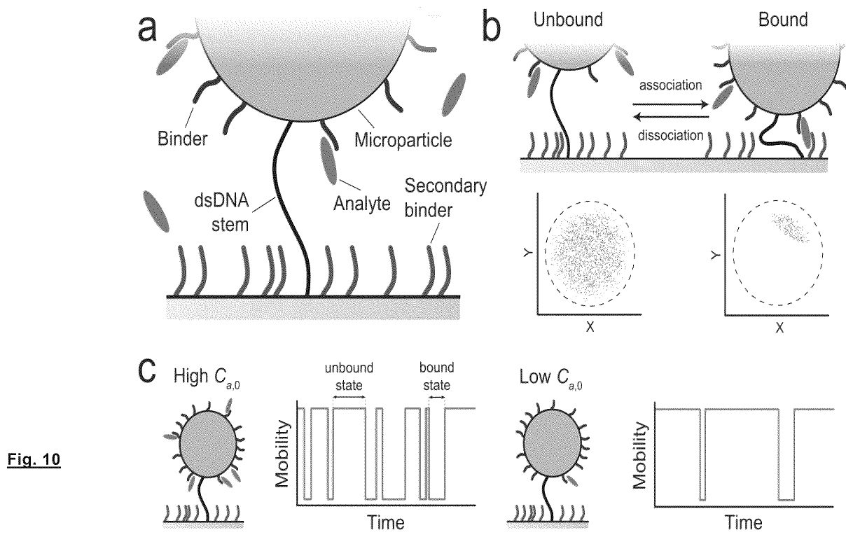

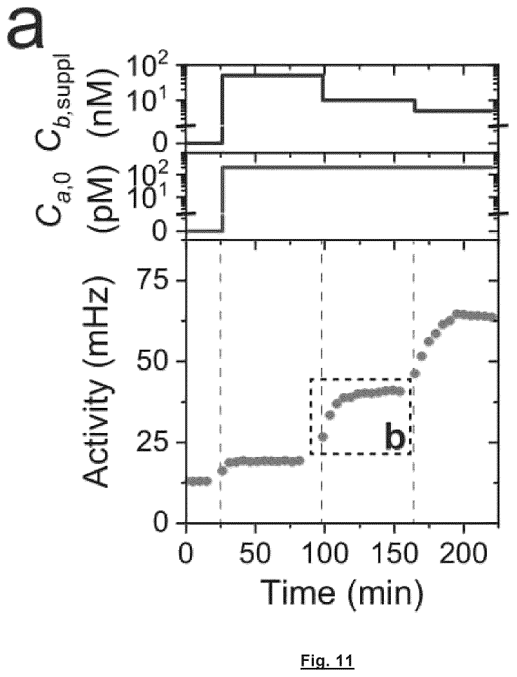

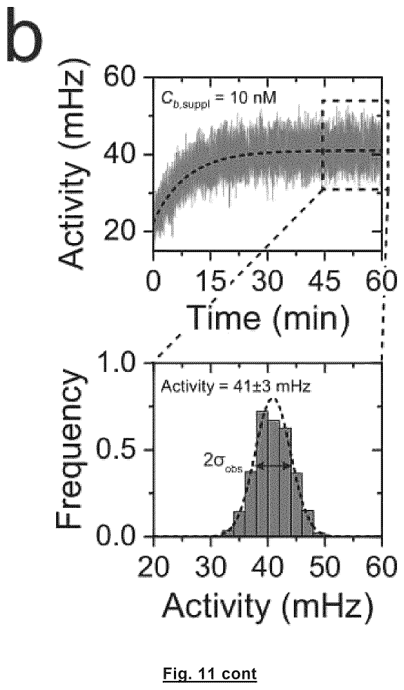

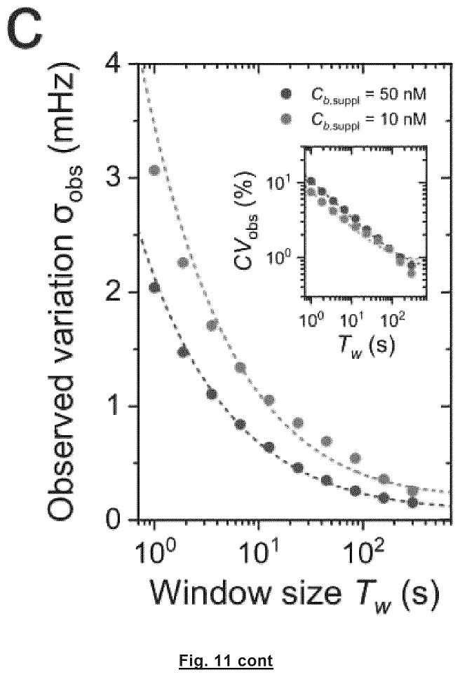

which equals the equilibrium value when an infinite volume is supplied (see Table 2). FIG. 3B. The fractional occupancy f as a function of time where cycles of analyte exchange and incubation are applied every 15 min with alternatingly C_{a,0}=0.15 pM and C_{a,0}=0.05 pM. The curve saturates at f_{eq.IV}=10·10⁻⁴, which equals the infinite-volume equilibrium value for the average concentration value C_{a,0}=0.1 μM. Dashed lines: continuous supply of C_{a,0}=0.05 pM yields f_{eq,IV}=5·10⁻⁴ and C_{a,0}=0.15 μM yields f_{eq,IV}=15·10⁻⁴. The right panel shows zoom-ins of three sections of the solid curve, each representing four cycles of instantaneous analyte exchange and subsequent incubations of 15 minutes. In zoom-in 1 (t=0-1 h) all curve segments show depletion behavior. In zoom-ins 2 (t=12-13 h) and 3 (t=42-43 h), depletion is seen for C_{a,0}=0.15 pM, since fᵢₙᵢₜ<f_{eq,IV}(C_{a,0}=0.15 pM), and repletion is seen for C_{a,0}=0.05 pM, since fᵢₙᵢₜ>f_{eq,IV}(C_{a,0}=0.05 pM). For all curve segments, the time-to-equilibrium τ=340 s. The vertical scale bars indicate Δf=10⁻⁴.

FIG. 4: experimental study of a limited-volume assay with varying supplemented binder concentrations using Biosensing by Particle Mobility (BPM)

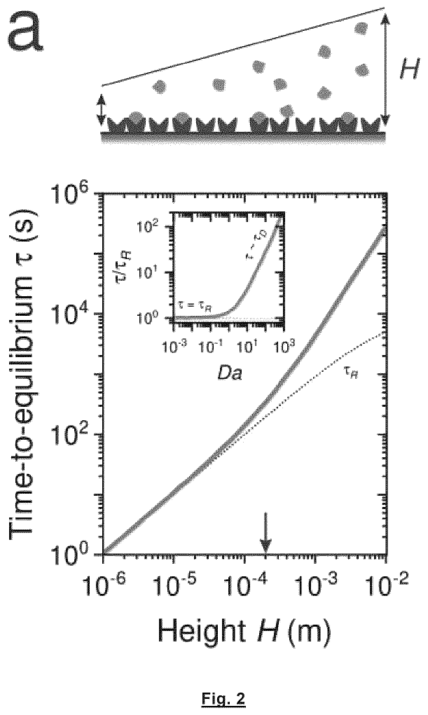

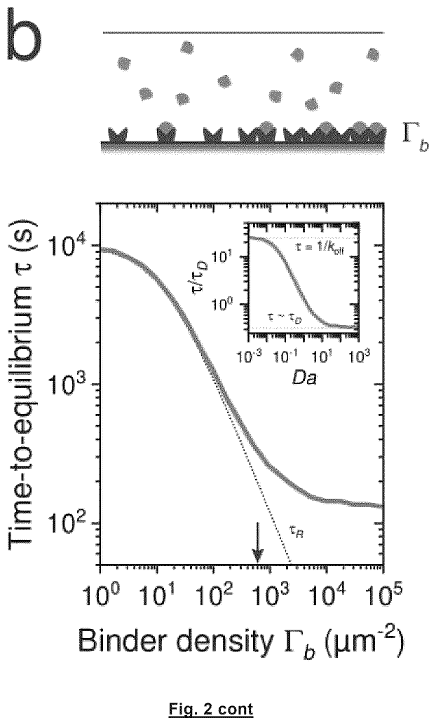

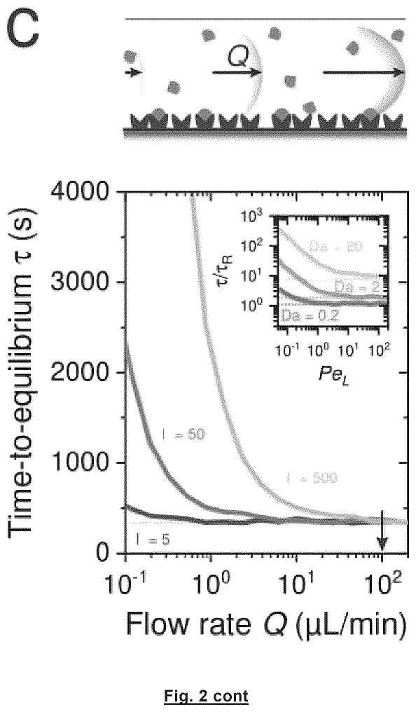

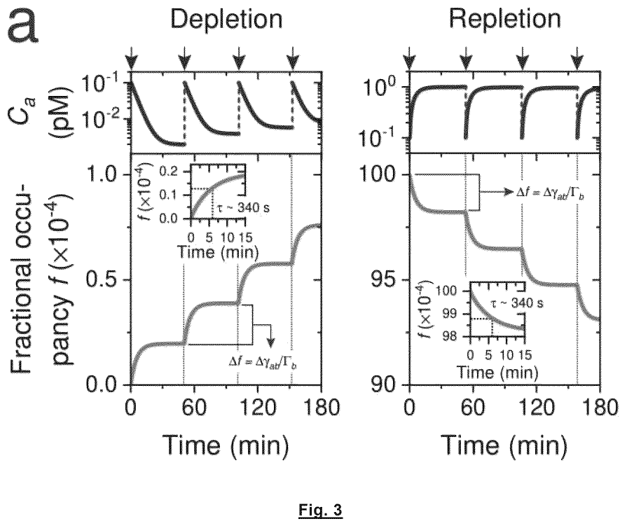

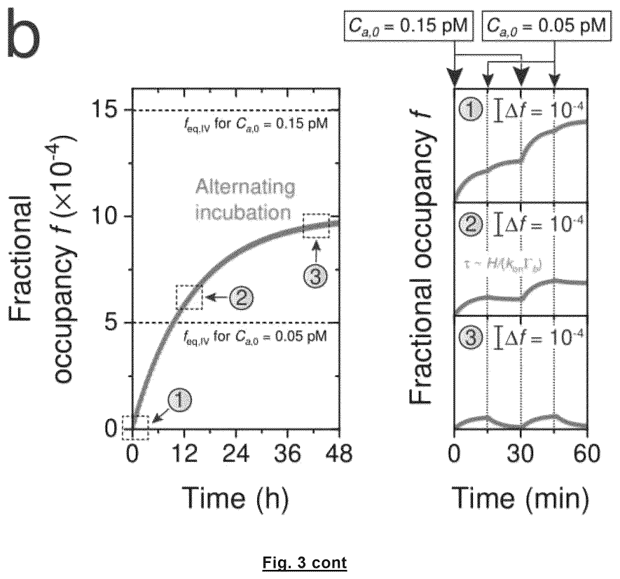

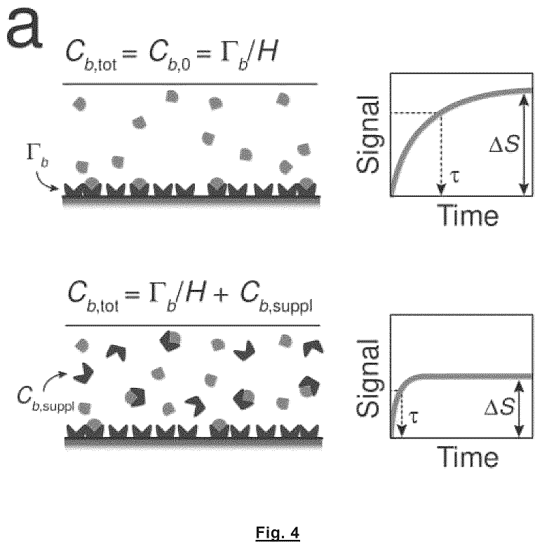

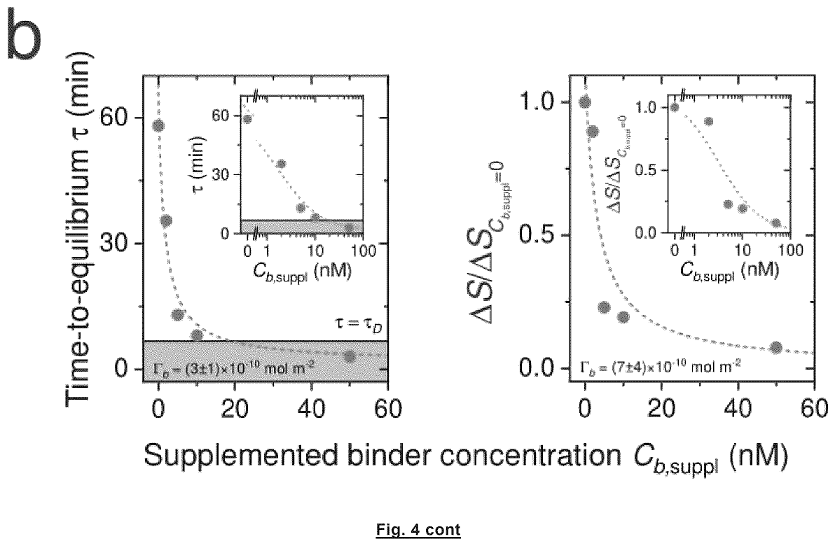

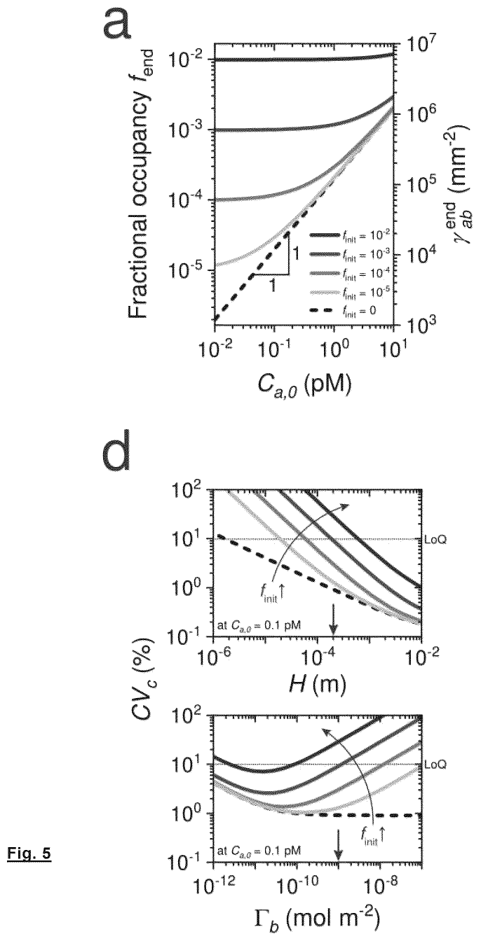

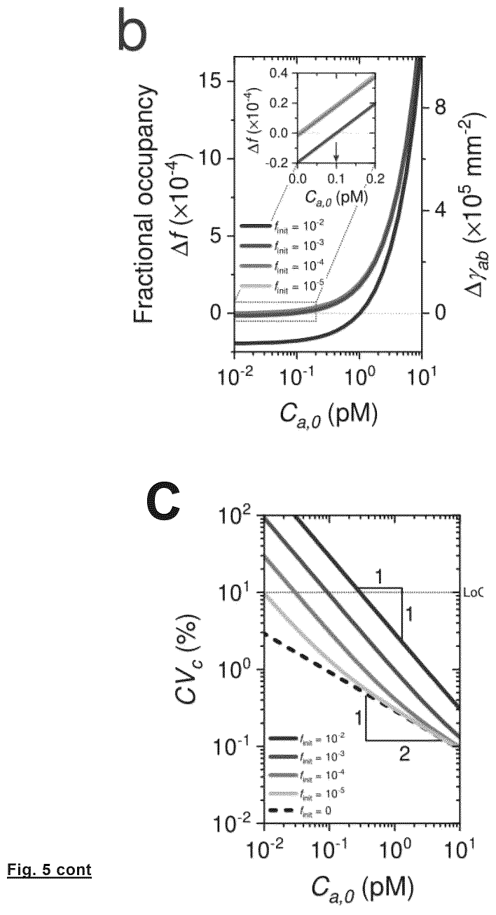

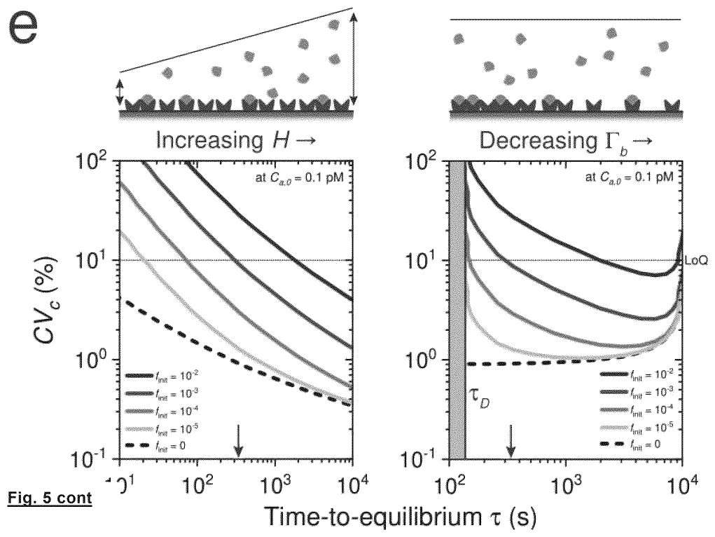

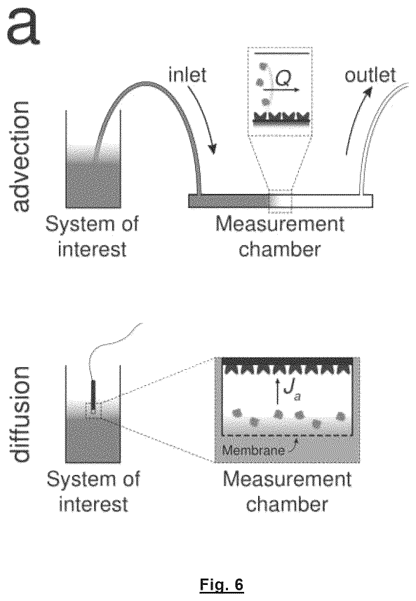

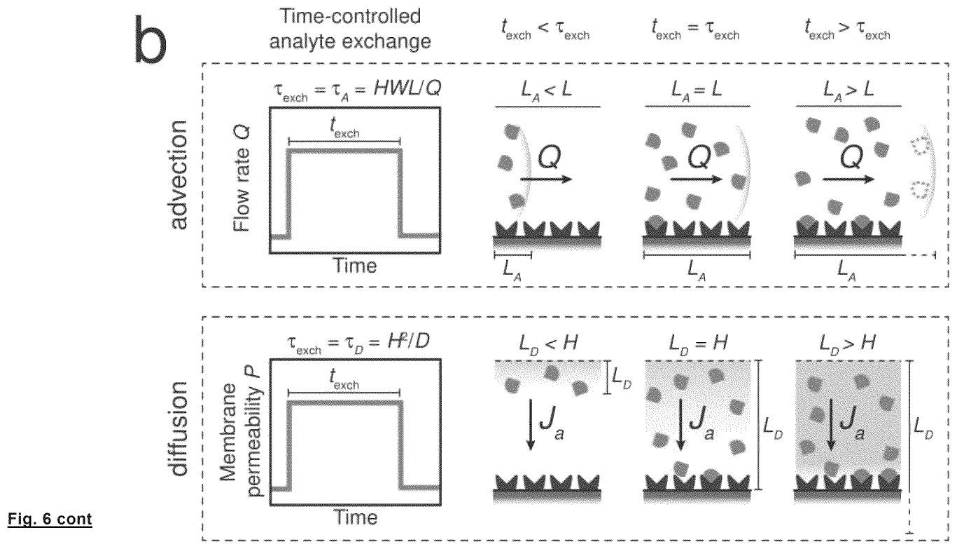

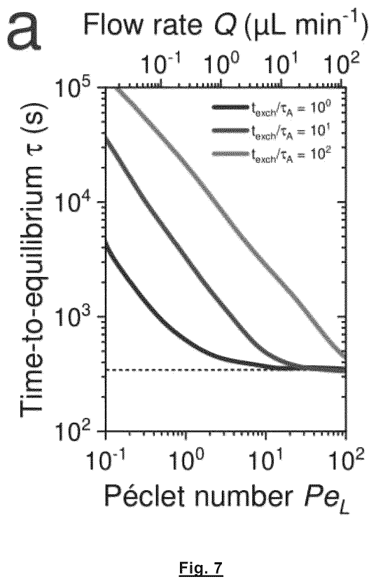

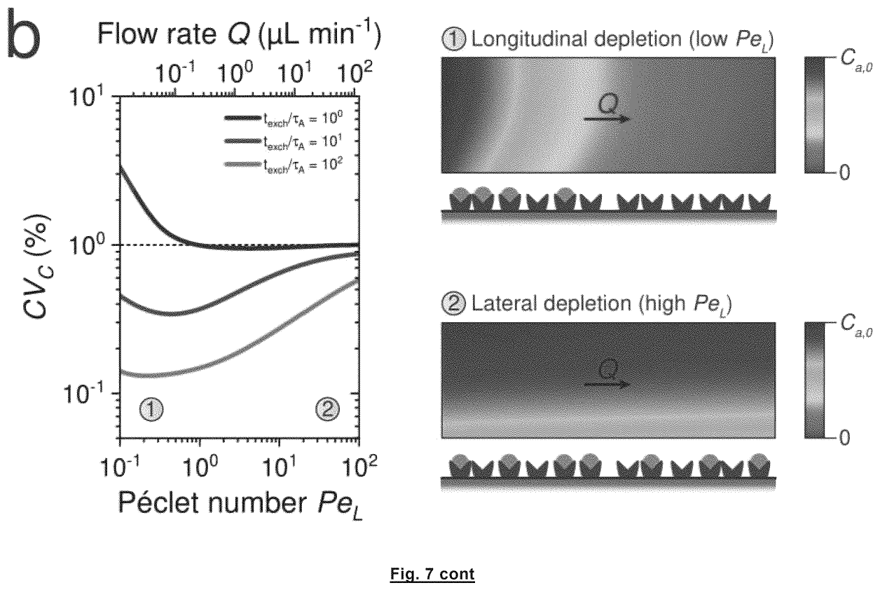

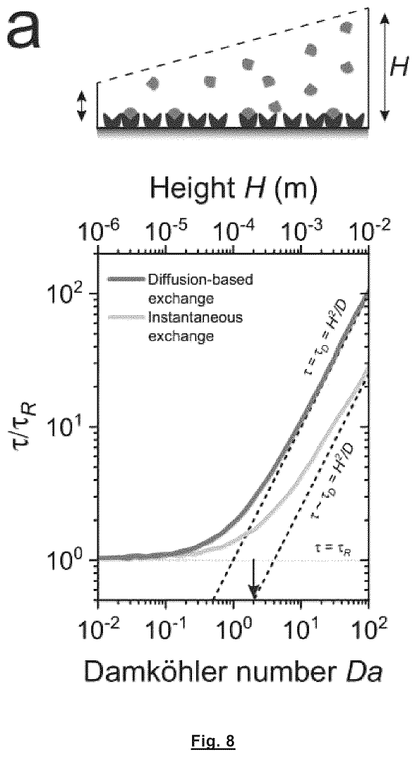

FIG. 4A. Sketch of the measurement chamber in a BPM measurement (see Supplementary Information 7) without (top) and with (bottom) supplemented binders with concentration C_{b,suppl}. For simplicity the particles of the BPM sensor are not shown in the sketch. In the absence of supplemented binders, the total binder concentration C_{b,tot} equals C_{b,tot}=Γ_{b}/H; in the presence of supplemented binders, the total binder concentration C_{b,tot} equals C_{b,tot}=Γ_{b}/H+C_{b,suppl}. Supplemented binders give a shorter time-to-equilibrium since the time-to-equilibrium scales according to τ_{R,LV}∝1/C_{b,tot} (see Table 1). Supplemented binders give a lower signal change because analytes captured in solution do not generate signal on the sensor surface. FIG. 4B. Experimentally observed time-to-equilibrium τ (left) and normalized signal change ΔS (right) as a function of supplemented binder concentration C_{b,suppl} in a BPM measurement with DNA-DNA hybridization reaction for an analyte concentration of 200 μM (see Supplementary Information 7). Left: the dashed line shows the fitted curve τ=p₁/(p₂+C_{b,suppl})+p₃, where p₁=1/kₒₙ (kₒₙ is assumed to be equal for all binders), p₂=Γ_{b}/H, and p₃ is the delay contributed by diffusion (see τ_{D}, black line, see also FIG. 2B) and experimental steps. Assuming H=200 μm (see Table 1), the fit gives Γ_{b}=(3±1)·10⁻¹⁰ mol m⁻², which is comparable to the standard parameter value as listed in Table 1. The fitted association rate constant is kₒₙ=(1.5±0.4)·10⁵ M⁻¹ s⁻¹, which is in the range of values reported in literature for comparable DNA-DNA hybridization reactions. Right: in the depletion condition (fᵢₙᵢₜ<f_{eq,IV}) the fractional occupancy scales according to f∝1/C_{b,tot}=C_{a,0}/(C_{b,ch}+C_{b,suppl}). The dashed line shows the fitted curve ΔS=p₁/(p₂+C_{b,suppl}), where p₁ scales the change in fractional occupancy to signal change and p₂=Γ_{b}/H. For H=200 μm, it was found that Γ_{b}=(7±4)·10⁻¹⁰ mol m⁻², which is comparable to the previously found value for Γ_{b} and the standard parameter value as listed in Table 1. The insets show the same data on lin-log scales. The errors reported in the Figure (smaller than the symbol size) and the caption are fitting errors based on a 68% confidence interval.

FIG. 5: analytical performance of the limited-volume assay, derived from simulations of a single measurement cycle

FIG. 5A. Fractional occupancy at the end of the incubation f_{end} as a function of analyte concentration C_{a,0} for different initial fractional occupancies fᵢₙᵢₜ. The right y-axis indicates the number of surface-bound analytes at the end of the cycle γ_{ab}^{end}. FIG. 5B. The absolute change of fractional occupancy Δf as a function of C_{a,0} for various fᵢₙᵢₜ. The right y-axis indicates Δγ_{ab}. A positive Δf and Δγ_{ab} indicate depletion; negative values indicate repletion. The inset shows the same data on a lin-lin scale. FIG. 5C. The coefficient of variation CV_{C} with which the analyte concentration C_{a,0} can be determined as a function of analyte concentration C_{a,0} for various initial fractional occupancies fᵢₙᵢₜ. CV_{C} scales as 1/\sqrt{C_{a,0}} for low fᵢₙᵢₜ and high C_{a,0}; CV_{C} scales as 1/C_{a,0} for high fᵢₙᵢₜ and low C_{a,0}. FIG. 5D. CV_{C} as a function of measurement chamber height H (top) and binder density τ_{b} (bottom) for various initial fractional occupancies fᵢₙᵢₜ and C_{a,0}=0.1 pM. The arrows on the x-axes indicate the standard parameter values for H and τ_{b} which as listed in Table 1. FIG. 5E. CV_{C} as a function of the observed time-to-equilibrium τ when varying the measurement chamber height H (left) or binder density τ_{b} (right) for various initial fractional occupancies fᵢₙᵢₜ and C_{a,0}=0.1 pM. The sketches above the graphs visualize a measurement chamber with an increasing height or a decreasing binder density. The arrows on the x-axes indicate the obtained time-to-equilibrium using the standard parameter values for H and τ_{b} as listed in Table 1.

FIG. 6: time-controlled analyte exchange by advection and diffusion

FIG. 6A. Concepts of analyte exchange by advection (top) and by diffusion (bottom) between a system of interest and a measurement chamber, using flow rate Q and molar flux Jₐ through a semi-permeable membrane respectively. FIG. 6B. Schematic visualizations of time-controlled analyte exchange by controlling the flow rate Q (top) and the molar flux Jₐ (by controlling the membrane permeability P, bottom) in time. The primary exchange has a characteristic time τ_{exch}, which equals τ_{A} for advection-based analyte exchange, and τ_{D} for diffusion-based analyte exchange, and a duration of t_{exch} in which analytes travel characteristic length L_{A} or L_{D}. Three regimes are identified: 1) t_{exch}<τ_{exch} where L_{A}<L or L_{D}<H, 2) t_{exch}=τ_{exch} where L_{A}=L or L_{D}=H, and 3) t_{exch}>τ_{exch} where L_{A}>L or L_{D}>H.

FIG. 7: the influence of advection-based analyte exchange on the performance of the analyte monitoring system using time-controlled analyte exchange by advection

FIG. 7A. The time-to-equilibrium τ as a function of the longitudinal Peclet number Pe_{L}, with the flow rate Q on the secondary x-axis (see Table 2), for three values of t_{exch}/τ_{A}. For small Pe_{L}, τ depends on Pe_{L} where τ∝1/Pe_{L} (see Note 7). τ decreases for increasing Pe_{L} since the primary exchange is faster (higher flow rate Q), but τ increases for increasing t_{exch}/τ_{A} since the duration of the primary exchange is longer. For large Pe_{L}, τ is independent of Pe_{L}; the Pe_{L} value where τ becomes independent of Pe_{L}, depends on the t_{exch}/τ_{A}. FIG. 7B. The precision of the measured concentration CV_{c} as a function of the longitudinal Peclet number Pe_{L} at an analyte concentration C_{a,0}=0.1 pM, with the flow rate Q on the secondary x-axis (see Table 2), for three values of t_{exch}/τ_{A}. The schematic visualizations of the measurement chamber cross-section show the spatial distribution of the concentration Cₐ, where red equals high Cₐ (Cₐ=C_{a,0}) and blue equals low Cₐ (Cₐ=0). For small Pe_{L}, a longitudinal depletion zone appears where (almost) all analytes are captured from solution by binder molecules directly after entering the measurement chamber, causing a positional dependency of the density of analyte-binder complexes, and a lower CV_{C} (i.e., a higher precision) at the point of sensing. For t_{exch}/τ_{A}=10⁰ this effect is largest since analyte exchange has a short duration. By increasing t_{exch}/τ_{A}, the CV_{C} decreases since more analytes are exchanged. For large Pe_{L}, a lateral depletion zone appears where the analyte exchange can be assumed to be instantaneous (see FIG. 2); here CV_{C} is independent of Pe_{L} and no positional dependency of the density of analyte-binder complexes exists.

FIG. 8: influence of diffusion-based analyte exchange on the observed time-to-equilibrium τ

FIG. 8A. Time-to-equilibrium τ normalized to the characteristic reaction time CR as a function of Damköhler number Da, with the measurement chamber height H on the secondary axis, for diffusion-based (orange) and instantaneous analyte exchange (light orange). For small Da, no difference in τ/τ_{R} exists since the observed reaction is reaction-limited. For large Da, diffusion-based analyte exchange results in a slower observed reaction since the characteristic distance L_{D} is larger. FIG. 8B. The time-to-equilibrium τ normalized to the characteristic diffusion time τ_{D} as a function of Damköhler number Da, with the binder density τ_{b} on the secondary axis. A difference in τ/τ_{D} exists caused by a longer L_{D}. For large Da, the observed reaction is diffusion-limited. In these simulations is was assumed that t_{exch}/τ_{D}=1. In both panels, the black arrows on the x-axis indicate the standard parameter value for Da (using H and Γ_{b}) which is given in Table 2.

FIG. 9: the influence of the primary exchange on the performance of the analyte monitoring system using time-controlled analyte exchange

FIG. 9A. Sketches of the time-evolution of the fractional occupancy upon analyte exchange by advection (by controlling flow rate Q) or diffusion (by controlling membrane permeability P). Three regimes are identified: 1) τ>t_{exch} where τ is determined by the mass transport by diffusion within the measurement chamber itself, 2) τ˜τ_{exch} where τ is determined by the duration of the primary exchange, and 3) τ<τ_{exch} where τ is determined by the dissociation rate constant k_{off}. FIG. 9B. Performance using time-controlled analyte exchange by diffusion for a sensor with parameters described in Table 2. Left: τ/τ_{D} as a function of t_{exch}/τ_{D}. For small t_{exch}/τ_{D} (where τ>t_{exch}), τ is independent of the exchange time since τ is limited by the mass transport after analyte exchange within the measurement chamber (reaction and diffusion, see FIG. 2A-FIG. 2B and FIG. 8). For large t_{exch}/τ_{D} (where τ<t_{exch}), τ is independent of the exchange time since the assay can be considered as an infinite-volume assay. When τ˜t_{exch}, τ is strongly determined by the duration of the primary exchange t_{exch}. The dashed black line represents τ=t_{exch}. Right: CV as a function of t_{exch}/τ_{D}. For small t_{exch}/τ_{D} (where τ>t_{exch}), CV_{C} depends on the amount of exchanged analytes where an increasing t_{exch} results in a decreasing CV_{C} where

$$C {V}_{C} ∝ \sqrt[4]{{t}_{e x c h}}$$

(dashed black line, see Note 8). For large t_{exch}/τ_{D} (where τ<t_{exch}), CV_{C} is independent of t_{exch} since the assay can be considered as an infinite-volume assay. When τ˜t_{exch}, CV_{C} depends more strongly on t_{exch} due to an increased molar flux Jₐ, where CV_{C}∝\sqrt{t_{exch}} (dashed black line, see Note 8). The black arrows on the x-axis indicate the value for t_{exch}/τ_{D}=1 used in FIG. 8. FIG. 9C. Performance using time-controlled analyte exchange by advection with τ/τ_{A} and CV_{C} as a function of t_{exch}/τ_{A}. Left: τ/τ_{A} as a function of t_{exch}/τ_{A}. The shape of the graph is similar to panel b, left, though shifted to higher values of t_{exch}/τ_{A} which depends on the flow rate which is used for analyte exchange. Besides, small t_{exch}/τ_{A} yield τ/τ_{A}≠1 due to an additional diffusion time penalty caused by the mass transport within the measurement chamber, during and after the primary exchange. The dashed black line represents τ=t_{exch}. Right: For small t_{exch}/τ_{A} (where τ>t_{exch}), CV_{C} is roughly independent of t_{exch} since the analyte exchange by advection includes an outlet where analytes are lost, in contrast to analyte exchange by diffusion. Therefore the minimum CV_{C} which can be reached theoretically by analyte exchange by advection (dashed black line, see Note 9) is much lower than the observed CV_{C}. For large t_{exch}/τ_{A} (where τ<t_{exch}), CV_{c} is independent of t_{exch} since the assay can be considered as an infinite-volume assay. No values for t_{exch}/τ_{A}<0 are shown for analyte exchange by advection, since in this regime positional dependency strongly influences τ and CV_{C}. The black arrows on the x-axis indicate the value for t_{exch}/τ_{A}=1 used in FIG. 7 and FIG. 2C.

FIG. 10: measurement principle of Biosensing by Particle Mobility (BPM)

FIG. 10A. Micrometer-sized particles (yellow) are tethered to a substrate using a dsDNA stem (black). The particle is functionalized with ssDNA binder molecules (brown) and the planar surface with ssDNA secondary binder molecules (light brown). Both binders can bind reversibly to single ssDNA analytes (orange) present in solution. FIG. 10B. Analytes binding to the binder molecules on the particle and subsequently the secondary binder molecules on the planar surface cause the particle to exhibit distinct Brownian motion patterns, i.e., the projection of the center of the particle onto the xy-plane, corresponding to an unbound state (high mobility) or a bound state (low mobility). FIG. 10C. Digital binding and unbinding events are identified by following the mobility of the particles over time. The time between two events corresponds to either the unbound state lifetime, or the bound state lifetime. For a high or low target concentration in solution, the microparticle shows a high or a low switching frequency respectively.

FIG. 11: the precision of Biosensing by Particle Mobility (BPM) measurements with time-controlled analyte exchange

FIG. 11A. The response of a BPM sensor with time-controlled analyte exchange. Activity per measurement of 5 minutes as a function of time for multiple consecutive measurement cycles (orange). At the start of each cycle (vertical lines), C_{a,0} was set to 200 pM (light brown) and a varying supplemented binder concentration C_{b,suppl} was added (dark brown). FIG. 11B. Top: zoom-in of the activity as a function of time calculated by a moving average with time window T_{w}=1 s, for C_{b,suppl}=10 nM (see panel a). The dashed line shows a single exponential fit (Sᵢₙᵢₜ+ΔS·e^{-t/τ}, see Supplementary Information 2), from which the time-to-equilibrium τ=474+2 s and the signal change ΔS=19.02±0.05 mHz could be determined (see FIG. 4B). Bottom: distribution of the observed activity at equilibrium (t>45 min). The dashed line is a fitted normal distribution with a mean activity A and an observed variation σ_{obs}. FIG. 11C. Observed variation σ_{obs} as a function of T_{w} for the first cycle (C_{b,suppl}=50 nM, brown) and second cycle (C_{b,suppl}=10 nM, orange). The dashed lines give the fit of the data according to Equation S14. For T_{W}→∞, the observed variation σ_{obs} approaches the variation induced by other sources than the measurement itself σₒₜₕₑᵣ, which equals σₒₜₕₑᵣ=0.09±0.02 mHz for C_{b,suppl}=50 nM and σ_{obs}=0.21±0.03 mHz for C_{b,suppl}=10 nM. The inset shows the same data, with the coefficient of variation CV_{obs} as a function of T_{W}. For T_{W}→∞, it was found that CV_{obs}=(7±2)·10⁻³ for C_{b,suppl}=50 nM and CV_{obs}=(9±1)·10⁻³ for C_{b,suppl}=10 nM. The errors reported in panel a are stochastic errors (smaller than the symbol size) while in panel c (smaller than the symbol size) and the caption of panel c, the reported errors are fitting errors based on a 68% confidence interval.

1-20. (canceled)

21. A method for the monitoring of an analyte of interest including at least one of a chemical, a biochemical, a biological substance, and a structure, present in or at a system of interest including at least one of a container, a reservoir, a reactor, a tube, a line, a vessel, a lumen, a tissue, an organ, and an organism, wherein a fluid or another viscoelastic medium or material comprises the analyte of interest, by measuring the concentration of the analyte of interest in a measurement chamber, wherein the measurement chamber comprises an effective number of binding sites (N_{b}), wherein the binding sites have a binding affinity to the analyte of interest, wherein the measurement chamber has an effective volume (V_{ch}) in which the analyte of interest has a significant probability to encounter the binding sites, and wherein the method comprises the step of providing a time-dependent sampling of the analyte of interest, by providing a time-dependent exchange of analyte between the system of interest and the effective volume (V_{ch}) of the measurement chamber, by performing at least one exchange modulation cycle comprising the following successive steps:
a) facilitating a primary exchange phase having a characteristic time of primary exchange (τ_{pr.exch.}) and a duration of primary exchange (t_{pr.exch.});
b) facilitating a primary-to-secondary switching phase having a characteristic primary-to-secondary switching time (σ_{pr.sec.switch}) and a primary-to-secondary switching duration (t_{pr.sec.switch}); and
c) facilitating a secondary exchange phase having a characteristic time of secondary exchange (τ_{sec.exch.}) and a duration of secondary exchange (t_{sec.exch.}),
wherein:
the exchange modulation cycle is repeated for any time-dependent sampling further provided;
the number of binding sites (N_{b}) and/or the effective volume (V_{ch}) of the measurement chamber is selected such that the effective volumetric binding site concentration (C_{b,ch}) in the measurement chamber is present in excess compared to the effective equilibrium dissociation constant (K_{d}) of the affinity binding between analyte of interest and binding sites, where C_{b,ch} is expressed as N_{b}/V_{ch};
the concentration of the analyte of interest is determined by direct or indirect measuring the time-development of the amount of analyte of interest bound to at least one or more binding sites; and
the direct or indirect measuring of the time-development of the amount of analyte of interest bound to at least one or more binding sites involves at least two measurements performed at different time-points in at least one exchange modulation cycle.

22. The method according to claim 21, wherein the binding sites are present on or in a supporting structure including at least one of a planar surface, a surface with concave or convex structure, a chemically and/or physically patterned surface, a particle, a polymer, and a porous matrix, and/or wherein the binding sites are present in the fluid or another viscoelastic medium or material comprising the analyte of interest.

23. The method according to claim 21, wherein:
the sum of the duration of primary exchange (t_{pr.exch.}) and the primary-to-secondary switching duration (t_{pr.sec.switch}) and the duration of secondary exchange (t_{sec.exch.}) is larger than a characteristic time-to-equilibrium (i) in the measurement chamber.

24. The method according to claim 21, wherein after step c), the exchange modulation cycle is repeated by performing the following step before step a):
d) facilitating a secondary-to-primary switching phase having a characteristic secondary-to-primary switching time (τ_{sec.pr.switch}) and a secondary-to-primary switching duration (t_{sec.pr.switch}).

25. The method according to claim 21, wherein the time-dependent sampling of the analyte of interest is effectuated by time-dependent exchange of analyte by diffusion, advection, or by another active or passive physicochemical analyte transport method, or by a combination thereof.

26. The method according to claim 21, wherein the duration of primary exchange (t_{pr.exch.}) is smaller than the characteristic incubation time-to-equilibrium (i) and/or the duration of primary exchange (t_{pr.exch.}) is larger than the characteristic time of primary exchange (τ_{pr.exch.}).

27. The method according to claim 25, wherein the duration of primary exchange (t_{pr.exch.}) is larger than one-hundredth of the characteristic time of primary exchange (τ_{pr.exch.}).

28. The method according to claim 21, wherein the primary-to-secondary switching duration (t_{pr.sec.switch}) is larger than the characteristic primary-to-secondary switching time (τ_{pr.sec.switch}), and/or the characteristic primary-to-secondary switching time (τ_{pr.sec.switch}) is smaller than the characteristic time-to-equilibrium (τ).

29. The method according to claim 21, wherein the secondary-to-primary switching duration (t_{sec.pr.switch}) is larger than the characteristic secondary-to-primary switching time (τ_{sec.pr.switch}), and/or the characteristic secondary-to-primary switching time (τ_{sec.pr.switch}) is smaller than the characteristic time-to-equilibrium (τ).

30. The method according to claim 21, wherein the sum of the duration of primary exchange (t_{pr.exch.}) and the primary-to-secondary switching duration (t_{pr.sec.switch}) is smaller than the characteristic time-to-equilibrium (τ).

31. The method according to claim 21, wherein the duration of the secondary exchange (t_{sec.exch.}) is smaller than the characteristic time of secondary exchange (τ_{sec.exch.}).

32. The method according to claim 21, wherein the at least one exchange modulation cycle comprises two or more exchange modulation cycles.

33. The method according to claim 21, wherein the at least one exchange modulation cycle comprises two or more exchange modulation cycles, and wherein the measuring of the time-development of the amount of analyte of interest bound to at least one or more binding sites involves at least two measurements performed at different time-points in at least one exchange modulation cycle.

34. The method according to claim 21, wherein the facilitating of the phases during the at least one exchange modulation cycle is performed by diffusion, advection, or by another active or passive physicochemical analyte transport method, or by a combination thereof.

35. The method according to claim 21, wherein the phases of the at least one exchange modulation cycle are effectuated by controlling the transport method in time.

36. The method according to claim 21, wherein the increase or decrease of the time-development of the amount of analyte of interest bound to at least one or more binding sites during an exchange modulation cycle depends on the amount of analyte of interest bound to at least one or more binding sites in said exchange modulation cycle and in a previous exchange modulation cycle.

37. The method according to claim 21, wherein the binding of the analyte of interest to a binding site is measured by:
a property of the analyte of interest including by at least one of charge, refractive index, fluorescence, luminescence, absorption, change of conformation, enzymatic activity, color, and mass; or
a signal from another object including at least one of a molecule, substance, particle, label, surface, and a combination thereof, by energy transfer, resonance, scattering, absorption, motion, charge, refractive index, fluorescence, luminescence, change of conformation, enzymatic activity, color, and mass; and
wherein the measurement involves binding, conversion, competition, inhibition, displacement, amplification, molecular cascade or sandwich formation, or a combination thereof.

38. A system for monitoring at least one analyte of interest, the system comprising:
a measurement chamber comprising a number of binding sites (N_{b}), wherein the binding sites are able to bind the analyte of interest, and wherein the measurement chamber has an effective volume (V_{ch}); and
at least one exchange port including at least one of a tube, a channel, an opening, a connector, a valve, a permeable or semipermeable material, and a membrane, for time-dependent sampling of the analyte of interest involving transport into and/or out of the measurement chamber;
wherein the system is configured to perform the method according to claim 21.

39. The system according to claim 38, wherein the system is configured to monitor:
one analyte of interest; or
multiple analytes of interest, wherein the measurement chamber comprises multiple binding sites, and wherein each of the multiple binding sites is able to bind a specific analyte of interest selected from the group of multiple analytes of interest to be monitored.

40. The system according to claim 39, wherein the system is configured to monitor multiple analytes of interest, and wherein the system is further configured to perform multiple methods in parallel, wherein each of the methods performed monitors one analyte of interest of the multiple of analytes of interest to be monitored.
